(19) United States
(12) Patent Application Publication
(10) Pub. No.: US 2024/0200130 A1
(43) Pub. Date: Jun. 20, 2024

(54) HIGHLY SENSITIVE IN VITRO ASSAYS TO DEFINE SUBSTRATE PREFERENCES AND SITES OF NUCLEIC-ACID BINDING, MODIFYING, AND CLEAVING AGENTS

(71) Applicant: The General Hospital Corporation, Boston, MA (US)

(72) Inventors: J. Keith Joung, Winchester, MA (US); Vikram Pattanayak, Wellesley, MA (US); Karl Petri, Cambridge, MA (US)

(21) Appl. No.: 18/402,501

(22) Filed: Jan. 2, 2024

Related U.S. Application Data
(62) Division of application No. 16/386,472, filed on Apr. 17, 2019, now Pat. No. 11,898,203.
(60) Provisional application No. 62/767,633, filed on Nov. 15, 2018.
(60) Provisional application No. 62/659,073, filed on Apr. 17, 2018.

(51) Int. Cl. C12Q 1/6874; C12N 15/10

(52) U.S. Cl. CPC C12Q 1/6874; C12N 15/1058; C12Y 305/04005

(57) ABSTRACT

Methods and compositions for performing highly sensitive in vitro assays to define substrate preferences and off-target sites of nucleic-acid binding, modifying, and cleaving agents.

### CLAIM OF PRIORITY

This application is a divisional of U.S. patent application Ser. No. 16/386,472, filed Apr. 17, 2019, which claims the benefit of U.S. Provisional Patent Application Serial Nos. 62/767,633, filed on Nov. 15, 2018; and 62/659,073, filed on Apr. 17, 2018. The entire contents of the foregoing are hereby incorporated by reference.

### FEDERALLY SPONSORED RESEARCH OR DEVELOPMENT

This invention was made with Government support under Grant No. HR0011-17-2-0042 awarded by the Defense Advanced Research Projects Agency (DARPA). The Government has certain rights in the invention.

### SEQUENCE LISTING

This application contains a Sequence Listing that has been submitted electronically as an XML file named “40978-0038005_UPDATEDSL_ST26.XML.” The XML file, created on Mar. 11, 2024, is 169,416 bytes in size. The material in the XML file is hereby incorporated by reference in its entirety.

### TECHNICAL FIELD

Provided herein are methods and compositions for performing highly sensitive in vitro assays to define substrate preferences and off-target sites of nucleic-acid binding, modifying, and cleaving agents.

### BACKGROUND

Off-target activity is a major challenge for the safe or effective use of proteins with customizable DNA-binding activities (including but not limited to homing endonucleases, zinc fingers, transcriptional activator-like effectors (TALEs), and CRISPR-Cas9 system proteins) in clinical, industrial, and research settings.

### SUMMARY

Provided herein are methods and compositions for performing highly sensitive in vitro assays to define substrate preferences and off-target sites of nucleic-acid binding, modifying, and cleaving agents.

Provided herein are methods for identifying double stranded DNA sequences that are cleaved, modified, or bound by an enzyme. The methods include (i) providing a plurality of linear dsDNA oligonucleotides of known sequences, each oligonucleotide having a 5′ end and a 3′ end and bearing at least two copies of a unique identifier sequence at or near both the 3′ and 5′ ends of the oligonucleotide, and common sequences that are present at the 5′ and 3′ ends in each one of the oligonucleotides in the plurality; (ii) incubating the plurality in the presence of an enzyme selected from site-specific nucleases, DNA modifying proteins, and DNA binding domains, under conditions sufficient for cleavage, modification, or binding to occur; (iii) selecting, and optionally enriching for, oligonucleotides that are cleaved, modified, or bound; and (iv) determining the sequences of the selected oligonucleotides that are cleaved, modified, or bound, thereby identifying double stranded DNA sequences that are cleaved, modified, or bound by an enzyme. Also provided herein are methods for identifying double stranded DNA sequences that are cleaved, modified, or bound by an enzyme. The methods include (i) providing an initial plurality of linear dsDNA oligonucleotides of known sequences, each oligonucleotide having a 5′ end and a 3′ end and bearing two copies of a unique identifier sequence at or near both the 3′ and 5′ ends of the oligonucleotide, and a common sequence that is present in each one of the oligonucleotides in the plurality; (ii) incubating the plurality in the presence of an enzyme selected from site-specific nucleases, modifying proteins, and DNA binding domains, under conditions sufficient for cleavage, modification, or binding to occur; (iii) selecting oligonucleotides that are not cleaved, modified, or bound; and (iv) determining the sequences of the selected oligonucleotides that are not cleaved, modified, or bound; and (v) comparing the sequences of the selected oligonucleotides that are not cleaved, modified, or bound to the sequences of the initial plurality of pre-enriched linear dsDNA oligonucleotides of known sequences; wherein the linear dsDNA oligonucleotides in the initial plurality that were not selected are identified as being cleaved, modified, or bound by the enzyme.

Further, provided herein are methods for identifying double stranded DNA sequences that are modified by a base editing enzyme (e.g., a cytidine deaminase that converts deoxycytidine to deoxyuridine or an adenine base editing enzyme that converts deoxyadenine to deoxyinosine). The methods include (i) providing a plurality of linear dsDNA oligonucleotides of known sequences, each oligonucleotide having a 5′ end and a 3′ end and bearing two copies of a unique identifier sequence at or near both the 3′ and 5′ ends of the oligonucleotide, and a common sequence that is present in each one of the oligonucleotides in the plurality; (ii) incubating the plurality linear dsDNA oligonucleotides in the presence of a base editing enzyme under conditions sufficient for modification to occur; (iii) amplifying the oligonucleotides with a polymerase that converts edited base pairs to equal mixtures of canonical base pairs (such as a uracil tolerant polymerase that converts dU:dG base pairs to equal mixtures of dT:dA and dC:dG base pairs, or dI:dT base pairs to equal mixtures of dA:dT and dG:dC base pairs) during DNA synthesis (i.e., wherein a dATP nucleotide is incorporated across from dU or a dCTP nucleotide is incorporated across from dI), such that an oligonucleotide that has been modified by the base editing enzyme will be amplified as a mixture of the original barcode-linked sequence from the pre-treatment library and also a modified sequence that contains substitutions (for example dC→dT or dA→dG); and (iv) determining the sequences of the amplified oligonucleotides, thereby identifying double stranded DNA sequences that are modified by the base editing enzyme.

Additionally, provided herein are methods for identifying double stranded DNA sequences that are modified by a cytidine deaminase base editing enzyme that converts cytidine to uridine and generates a nick on the opposite strand. The methods include (i) providing a plurality of linear dsDNA oligonucleotides of known sequences, each oligonucleotide having a 5′ end and a 3′ end and bearing two copies of a unique identifier sequence at or near both the 3′ and 5′ ends of the oligonucleotide, and a common sequence that is present in each one of the oligonucleotides in the plurality; (ii) incubating the plurality of linear dsDNA oligonucleotides in the presence of a base editing enzyme under conditions sufficient for modification to occur, and then incubating the plurality of linear dsDNA oligonucleotides in the presence of enzymes to generate a single-strand break (nick) at sites with uridine nucleotides, thereby creating dsDNA oligonucleotides that contain two nicks with 5′ phosphates on opposite strands, thereby creating overhangs; (iii) incubating the dsDNA oligonucleotides with a DNA polymerase that creates 5′ phosphorylated blunt ends from the overhangs (e.g., T4 DNA polymerase or Phusion DNA polymerase or Phusion U DNA polymerase); (iv) capturing the phosphorylated blunt ends with double stranded DNA adapters comprising primer sequences; (v) amplifying the sequences using one primer specific to the adapter and one primer specific to the common sequence backbone; (vi) optionally performing an additional selection by performing size selection for smaller, cut fragments before or after amplification; and (iv) determining the sequences of the amplified oligonucleotides, thereby identifying double stranded DNA sequences that are modified by the base editing enzyme.

Further, provided herein are methods for identifying double stranded DNA sequences that are modified by an adenine base editing enzyme that converts deoxyadenine to deoxyinosine and generates a nick on the opposite strand. The methods include (i) providing a plurality of linear dsDNA oligonucleotides of known sequences, each oligonucleotide having a 5′ end and a 3′ end and bearing two copies of a unique identifier sequence at or near both the 3′ and 5′ ends of the oligonucleotide, and a common sequence that is present in each one of the oligonucleotides in the plurality; (ii) incubating the plurality of linear dsDNA oligonucleotides in the presence of a base editing enzyme under conditions sufficient for modification to occur, and then incubating the plurality of linear dsDNA oligonucleotides in the presence of endonuclease V enzymes to generate a single-strand break (nick) at sites with inosine nucleotides, thereby creating dsDNA oligonucleotides that contain two nicks with 5′ phosphates on opposite strands, thereby creating overhangs; (iii) incubating the dsDNA oligonucleotides with a DNA polymerase that creates 5′ phosphorylated blunt ends from the overhangs (e.g., T4 DNA polymerase or Phusion DNA polymerase or Phusion U DNA polymerase); (iv) ligating the phosphorylated blunt ends with double stranded DNA adapters comprising primer sequences; (v) amplifying the sequences using one primer specific to the adapter and one primer specific to the common sequence backbone; (vi) optionally performing an additional selection by performing size selection for smaller, cut fragments before or after amplification; and (iv) determining the sequences of the amplified oligonucleotides; thereby identifying double stranded DNA sequences that are modified by the base editing enzyme.

A method of identifying double stranded DNA sequences that are modified by an adenine base editing enzyme that converts deoxyadenine to deoxyinosine and generates a nick on the opposite strand or a cytidine deaminase base editing enzyme that converts cytidine to uridine and generates a nick on the opposite strand, the method comprising: (i) providing a plurality of linear dsDNA oligonucleotides of known sequences, each oligonucleotide having a 5′ end and a 3′ end and bearing two copies of a unique identifier sequence at or near both the 3′ and 5′ ends of the oligonucleotide, and a common sequence that is present in each one of the oligonucleotides in the plurality; (ii) incubating the plurality of linear dsDNA oligonucleotides in the presence of Endonuclease MS from Thermococcus kodakarensis (TkoEndoMS) to induce double strand breaks (DSBs) at deamination sites in the substrate DNA to produce DNA fragments with single-stranded, 5 base pair overhanging ends centered at the deamination site; (iii) treating the DNA fragments with uracil DNA glycosylase and endonuclease VIII to remove the deoxyuracil base from the ends of the DNA fragments; (iv) end-repairing and/or A-tailing the ends of the DNA fragments; (v) ligating an adapter oligonucleotide (preferably comprising sequences for use in high throughput sequencing) to the end; and (vi) sequencing the DNA fragments. In addition, provided herein are methods for identifying double stranded DNA sequences that are bound by a catalytically-inactive Cas9 in the presence of a selected gRNA or another DNA-binding domain. The methods include (i) providing a plurality of linear dsDNA oligonucleotides of known sequences, each oligonucleotide having a 5′ end and a 3′ end and bearing two copies of a unique identifier sequence at or near both the 3′ and 5′ ends of the oligonucleotide, and a common sequence that is present in each one of the oligonucleotides in the plurality; (ii) incubating the plurality in the presence of a DNA binding domain, e.g., Cas9 enzyme complexed with sgRNAs or another DNA-binding domain, that is attached to magnetic beads (e.g., covalently bound or bound by an affinity handle), under conditions sufficient for binding to occur; (iii) selecting, and optionally enriching for, oligonucleotides that are bound through one or more sets of bead pulldown and washing in an appropriate buffer to promote dissociation into supernatant of unbound molecules, followed by elution of bound DNA either in an appropriate buffer to promote dissociation of any bound DNA or in a buffer containing a protease, such as proteinase K, to degrade bead-bound protein and release bound DNA; and (iv) determining the sequences of the selected oligonucleotides that are cleaved, thereby identifying double stranded DNA sequences that are bound by the DNA binding domain.

In some embodiments, the linear dsDNA oligonucleotides used in methods described herein include (i) a set of all potential off-target sequences in a reference genome bearing up to a certain number of mismatches relative to an identified on-target site (analogous to genomic DNA libraries); (ii) a comprehensive set of potential off-target sites bearing up to a certain number of mismatches (analogous to random base substitution libraries); (iii) a library of potential off-target sequences present in a set of variant genomes from defined populations (i.e., genomic DNA libraries designed to reflect DNA sequence variants present in a population of individuals); or (iv) another relevant defined set of potential off-target sites (for example, oncogene hotspots or sequences from tumor suppressor genes).

In some embodiments, the pre-enriched linear DNA library members are first synthesized as individual single-stranded DNA sequences, e.g., on high-density oligonucleotide arrays; and the single-stranded DNA sequences are converted into double-stranded DNA molecules by priming against the common sequence, optionally before or after being released from the chip.

In some embodiments, the pre-enriched linear DNA library members represent 1) a set of all potential off-target sequences in a reference genome bearing up to a certain number of mismatches relative to the on-target site (analogous to genomic DNA libraries), 2) a comprehensive set of potential off-target sites bearing up to a certain number of mismatches (analogous to random base substitution libraries), 3) a library of potential off-target sequences present in a set of variant genomes from defined populations (i.e., genomic DNA libraries designed to reflect DNA sequence variants present in a population of individuals), or 4) other relevant defined sets of potential off-target sites (for example, oncogene hotspots or sequences from tumor suppressor genes).

In some embodiments, the pre-enriched linear DNA library members comprise at least 1,000, 2500, 5000, or 10,000 and up to 10⁶, 10⁷, 10⁸, 10⁹, 10¹⁰, or 10¹¹ different sequences, e.g., 10-100k different sequences.

In some embodiments, the pre-enriched linear DNA library members comprise sequences that are 50-500, e.g., 100-400, e.g., 150 to 300 bp long, e.g., 200 to 280 bp long. Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1. Illustration of the differences in library complexity among base substitution library (SEQ ID NOs.: 1-6, 1, and 7-11) and genomic DNA libraries (SEQ ID NOs.: 1 and 12-16). For genomic libraries, the on-target site (in a red box in this example) retains very little similarity to the other roughly ˜3 billion genomic sequences. For base substitution libraries, the pre-selection libraries are enriched for sites that are not necessarily present in the genome but that are similar to the intended target site. Substitutions are indicated by lower case letters.

FIG. 2. Illustrative overview of exemplary method. A user-defined set (typically, but not limited to, 10,000 to 100,000 sequences) of potential DNA substrates is generated by synthesis on a high-density oligonucleotide array and then made double-stranded. Three potential examples of sets of sequences to use include the set of all sequences with up to six mismatches in the human genome, eight mismatches in the human exome, or all possible DNA sequences with up to three mismatches. The double-stranded DNA library can then be used in screens for sequence modification, screens for sequence depletion, or selection for sequence modification and/or cleavage. Black lines indicate constant sequence that is present in every member of the library and is used for primer binding sites during amplification steps and in bioinformatic processing.

FIG. 3. Target site libraries can be selected for cleavage (strategy 1) or screened for depletion of cleaved sequences (strategy 2). Black lines indicate constant sequence that is present in every member of the library and is used for primer binding sites during amplification steps and in bioinformatic processing. SEQ ID NOs.: 17-22 are shown.

FIG. 4. Enrichment of Cas9 cleavage sites by selection from a random base substitution library. The intended target site (SEQ ID NO:23) is listed below the heat map. Each black box represents the abundance of a particular nucleotide (denoted on left) at the position corresponding to the target site nucleotide listed below, with black representing the most abundant nucleotides/per position and white representing no abundance.

FIG. 5. Identification of on-target sequences through screening of random base substitution library and of genomic DNA library. Substrates with fewer numbers of mutations (indicated by lower m numbers, where (m_d_i)→m=number of mutations, i=number of insertions, d=number of deletions) in the substrate profiling library and in the genome-inspired library where Xm0 denotes m mismatches without any insertions, RNAmd indicates a target site deletion of length d with m mismatches at the remaining base pairs in the site, DNAmi indicates a target site insertion of length I with m mismatches at the remaining base pairs in the site).

FIG. 6. Compositions of representative genomic DNA oligonucleotide libraries are shown. The number of individual genomic sites are listed according to number of mismatches and bulges. Unless otherwise specified, these libraries were utilized in the experiments outlined in the subsequent figures.

FIG. 7. Library characterization. Uniformity metrics and drop out percentages are shown for each of the libraries, after oligonucleotide synthesis, library amplification, and Illumina sequencing. 90th percentile sequencing counts refer to the number of sequencing reads obtained for the library member in the 90th percentile, when ordered in terms of increasing reads. 90/10 ratio is the ratio of the number of sequencing reads for the 90th percentile library member divided by the 10th percentile library member and is a metric of library uniformity. Drop out refers to the number of sequences that were not represented in the sequenced, amplified libraries.

FIG. 8. Enrichment of known GUIDE-seq sites. Swarm plots are shown for representative cleavage selections using a method described herein (an example of which is referred to as the ONE-seq method). Each circle represents the aggregate read counts, normalized to the on-target sequence for a given guide RNA selection (listed on top), for an individual library member. The black stars indicate the on-target library member. Filled circles denote sites that were identified by published GUIDE-seq experiments. There were no published RNF2 GUIDE-seq sites.

FIG. 9. Enrichment of highly enriched CIRCLE-seq sites. Swarm plots are shown for representative cleavage selections using the ONE-seq method. Each circle represents the aggregate read counts, normalized to the on-target sequence for a given guide RNA selection (listed on top), for an individual library member. The black stars indicate the on-target library member. Filled circles denote sites with >100 read counts that were identified by published CIRCLE-seq experiments. There were no published RNF2 GUIDE-seq sites.

FIG. 10. Enrichment of moderately enriched CIRCLE-seq sites. Swarm plots are shown for representative cleavage selections using the ONE-seq method. Each circle represents the aggregate read counts, normalized to the on-target sequence for a given guide RNA selection (listed on top), for an individual library member. The black stars indicate the on-target library member. Filled circles denote sites with 10-99 read counts that were identified by published CIRCLE-seq experiments.

FIG. 11. Enrichment of lowly enriched CIRCLE-seq sites. Swarm plots are shown for representative cleavage selections using the ONE-seq method. Each circle represents the aggregate read counts, normalized to the on-target sequence for a given guide RNA selection (listed on top), for an individual library member. The black stars indicate the on-target library member. Filled circles denote sites with 1-9 read counts that were identified by published CIRCLE-seq experiments.

FIG. 12. Venn diagrams showing that the exemplary method (darker circles) identifies all 60 out of 62 highly enriched (>100 reads) CIRCLE-seq sites (lighter circles) of six SpCas9: sgRNA, using a cutoff of 1% of the on-target ONE-seq aggregated read count. The 2 sites that were not above the 1% ONE-seq cutoff do not necessarily represent bona fide off-target sequences and may be false positives in the CIRCLE-seq method. CIRCLE-seq does not identify 478 of the ONE-seq identified sites for these guide RNAs.

FIG. 13. Validation results of three FANCF off-target sites identified by ONE-seq but not GUIDE-seq or CIRCLE-seq. Targeted amplicon sequencing was performed on three of the five most highly enriched novel off-target candidates identified by ONE-seq from HEK293T cells sorted for the the top decile of expression of a SpCas9:FANCF sgRNA construct. On the left, total number of indel-containing (edited) sequence reads and total number of reference reads are shown, along with edit percentage and unedited candidate off-target sequence are shown. To the right, individual data from three separate sorting and control (untreated) experiments are shown. (SEQ ID NOs:24-39 appear in order) FIG. 14. Reproducibility of enrichment scores in variant libraries. ONE-seq selections were performed on an EMX1 genomic off-target library and an EMX1 genomic variant off-target library. Enrichment scores (relative to the on-target sequence) are shown for the library members that are shared by both libraries. The superimposed line corresponds to equal enrichment scores from both selections.

FIG. 15. ONE-seq identifies candidate off-target sites that are present in the population but not in the reference genome. Normalized aggregate read counts (where 1.0 is the on-target site) are shown for off-target candidates identified from the reference genome and paired off-target candidates that contain SNPs found in the 1000 genomes population. Variants that are present in >40% of the population are shown with filled circles. The superimposed line corresponds to equal enrichment scores from both paired library members.

FIG. 16. Base editor screening strategy. A random base substitution library designed for the EMX1 target site was incubated in vitro with BE1, and amplified by PCR with Kapa HiFi Uracil+DNA polymerase, which converts U:G base pairs to equal mixtures of T:A and C:G base pairs during DNA synthesis (a dATP nucleotide is incorporated across from dU). Therefore, any library member that can be modified by BE1, when sequenced, will sequence as a mixture of the original barcode-linked sequence from the pre-treatment library and also a modified sequence that contains C→T substitutions (and other rarer substitutions).

FIG. 17. Base editor screen demonstrates enrichment of sites containing NGG and demonstrates high specificity at the PAM-proximal end of the target site (SEQ ID NO:23) and lower specificity at the PAM distal end. Heat map is interpreted in the same way as in FIG. 4.

FIG. 18. BE3 selection strategy. In this strategy, target site libraries are exposed to BE3 enzyme and are enriched for modified members through double-strand break creation at sites with uridine nucleotides (through USER) and nicks (through BE3).

FIG. 19. Enrichment of BE3 off-target sites by ONE-seq. Normalized aggregate read counts (where 1.0 corresponds to the on-target site) are shown for eight ONE-seq selections on genomic DNA-inspired libraries. Only sites with a score of 0.01 or greater (1% of on-target enrichment) are shown. Black stars represent on-target library members. Filled in black circles denote newly validated off-target sites compared to Digenome-seq (with the exception of ABE Site 18, which was not tested by Digenome-seq). Open black circles denote Digenome-seq candidate sites.

FIG. 20. Newly identified and validated BE3 off-target sites. Data from targeted amplicon sequencing from genomic DNA from HEK293T cells expressing the indicated BE3:sgRNA complexes are shown, in comparison to an untreated control. Experiments were performed in three replicates. Only the 28 newly identified and validated BE3 off-target sites compared to Digenome-seq are shown.

FIG. 21. ABE selection strategy. In this example, the Adenine base editor (ABE) is used. ABE creates A→I changes in DNA. The method we use here to define off-target sites of ABE is similar to that used in Example 3, except a different endonuclease, endonuclease V, is used to create a nick at a deoxyinosine site in DNA.

FIG. 22. ABE selection on a base substitution library. Heatmap is interpreted in same way as in FIG. 4. The data from the selection demonstrate enrichment of NGG PAMs, but also enrichment of sequences containing an A at position five, relative to the SpCas9 cleavage selection in FIG. 4 and the BE3 selection in FIG. 8, demonstrating the need for an A in a certain editing window for ABE to demonstrate activity. (SEQ ID NO:23) FIG. 23. Enrichment of ABE7.10 off-target sites by ONE-seq. Normalized aggregate read counts (where 1.0 corresponds to the on-target site) are shown for eight ONE-seq selections on genomic DNA-inspired libraries. Black stars represent on-target library members. Filled circles denote validated off-target sites. Open dark circles denote off-target candidates that were sequenced in the validation study.

FIG. 24. Validated ABE off-target sites. Data from targeted amplicon sequencing from genomic DNA from HEK293T cells expressing the indicated ABEmax:sgRNA complexes are shown, in comparison to an untreated control. Experiments were performed in three replicates.

FIG. 25. Capillary electrophoresis data from an experiment demonstrating the specificity of TkoEndoMS' endonuclease activity for G:U DNA mismatches in vitro. An 800 base pair PCR amplicon was incubated with purified BE protein and a variable sgRNA for two hours to induce site-specific deamination. After purification, the deaminated PCR amplicon was incubated with purified TkoEndoMS protein for 7 minutes to induce double strand breaks at G:U mismatches. The DNA was then separated by size by capillary electrophoresis and imaged.

FIG. 26. Overview of enrichment for binding sites by pulldown. In this method, dCas9 coated beads are incubated with a library of potential off-target sites. Library members that are not bound are washed into supernatant, and bound library members are eluted by digesting bead-bound protein with Proteinase K. The resulting eluted library can either be amplified and subjected to additional rounds of pulldown or subjected to analysis by high throughput sequencing.

FIG. 27. Pulldown conditions can discriminate between on and off target sites. A mixture of three double stranded DNAs with differing lengths was subjected to binding site pulldown with dCas9:EMX1 sgRNA-coated beads. The on target site was present on a 280 base pair DNA, and one of two off target sites (OT2 or OT4) were present on a 220 base pair DNA. A third 200 bp (“random”) DNA containing neither on nor off-target site was also in the mixture. Enriched DNA was run on a QIAxcel. Lane A3 denotes a size ladder. Lane A1 shows selective pulldown of the 280 base pair on target site, but no pull down of the OT2 site or the 200 bp DNA. Lane A2 shows that the OT4 site can still be bound in the method, demonstrating that the conditions used in the pulldown are able to enrich for off target sequences that are capable of being bound by a dCas9: EMX1 sgRNA. (SEQ ID NOs:23, 40-41 appear in order)

FIG. 28. Enrichment of genomic DNA-inspired library by pulldown. A pulldown conducted in the presence of 50 ug/ml heparin on the FANCF library leads to enrichment of the on-target site (black star) to the most abundant post-pulldown library member.

FIG. 29. Post-selection library composition for I-PpoI. A sequence logo is shown for sequences with at least 1% of the normalized read counts of the intended I-PpoI target site. Position in the site from 5′ to 3′ end is shown on the horizontal axis, and the height of the stack of letters denotes information content (in bits) for each position. The height of each individual nucleotide highlights the relative contribution oft hat nucleotide to the information content oft hat position. Positions 2, 13, and 14 are the most highly specified (highest information content). Position 15 is the least specified (lowest information content).

### DETAILED DESCRIPTION

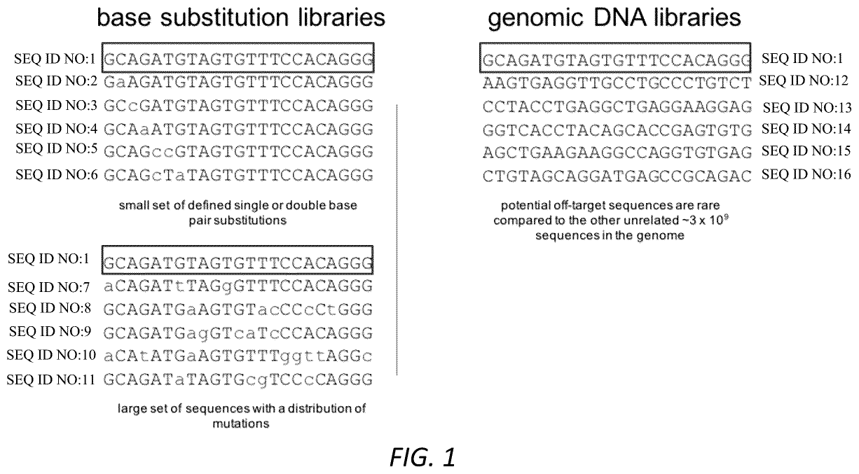

In vitro/biochemical strategies to understanding on- and off-target activity of DNA binding domains generally fall into two types (FIG. 1): In the first type, a set of DNA sequences in a relevant defined system (for example, the human genome) is interrogated for off-target cleavage events. Examples of methods that utilize this strategy include CIRCLE-seq (Tsai et al., Nat Meth. 14: 607 (2017)], SITE-seq (Cameron et al. Nat Meth. 14: 600 (2017)), and Digenome-seq (Kim et al. Nat Meth. 12: 237 (2015)). The scope of these genome-wide methods is restricted to off-target sites that are present in the particular genomic DNA used in the study. In contrast, in the second type of strategy, the substrate preference of the DNA binding/modifying protein is assayed in a more unbiased fashion by comprehensively interrogating a library of binding sites in which certain base positions are randomly substituted with all potential alternative bases (rather than just a limited set of base substitutions as in the case of the first strategy). These “genomic DNA” and “random base substitution library” approaches have been carried out for various nucleases (ZFNs (Pattanayak et al. Nat Meth. 8: 765 (2011)], TALENs (Guilinger et al. Nat Meth. 11: 429 (2014)), and CRISPR-Cas9 (Pattanayak et al. Nat Biotech. 31: 839 (2013))) in vitro and provide insights into the biochemical function and specificity of these nucleases.

Both types of strategies to study off-target activity have limitations that affect their abilities to identify bona fide off-target sites. In genome-wide selections, the tens to hundreds of cleaved off-target sites must be enriched from a background of billions of other sites that are not cleaved (the human genome has a length of ˜3 billion base pairs and therefore contains ˜6 billion sites to be assayed). For example, due to noise in the enrichment method and in the sequencing results, the CIRCLE-seq method is limited to detection of sites that have no more than six mismatches relative to the on-target site, which represents only ˜0.002% of the genomic material present in the assay. While some methods, such as Digenome-seq, rely on massive over sequencing of nuclease treated DNA libraries, methods like CIRCLE-seq and GUIDE-seq typically incorporate an enrichment step for edited sequences. This enrichment step can be performed in cells (GUIDE-seq) or in vitro (CIRCLE-seq). Although it is substantially more sensitive than other methods for off-target screening, the CIRCLE-seq method requires a very large input of genomic DNA (25 ug) for each experimental sample.

In vitro selections on unbiased base substitution libraries are limited by library size (the set of sequences that can practically be assayed). For example, an SpCas9 target site contains 22 potentially-specified base pairs (20 from hybridization to the guide RNA and two from the PAM sequence). To assay all potential target sites bearing all possible combinations of base substitutions at all positions, at least 4²²˜10¹³ unique molecules of DNA, would need to be generated and interrogated, neither of which is possible using current technologies. For example, library construction methodologies are currently limited to producing 10¹¹-10¹² unique molecules of DNA. Furthermore, even if library construction methodologies were improved, it is not feasible to sequence 10¹² molecules of DNA. To overcome this restriction, doped oligonucleotide synthesis is traditionally used to create a library of sites bearing base substitutions that follow a binomial distribution, such that the on-target site is present in more copies than each variant site in the library bearing a single mutation, each of which are present in more copies than each variant site in the library bearing double variant site, and so forth. Therefore, selections performed with these random base substitution libraries are limited by the fact that 1) it is not possible to create a completely unbiased library (i.e., they are heavily biased towards the intended on-target site sequence) and 2) it is not possible to create a library that uniformly represents the potential sequence space. Furthermore, using the outputs from defined libraries assays to predict or identify off-target sites in genomic sequences often requires extrapolation (Sander et al. Nucleic Acids Res. 41: e181 (2013)), because not all relevant genomic sequences are guaranteed to be covered in pre-selection (limited to 10¹² sequences, which corresponds to six or seven substitutions) or post-selection libraries (limited to 10⁷⁻⁸ sequences by sequencing capacity).

### Methods of Identifying DNA Binding, Modification, or Cleavage Sites

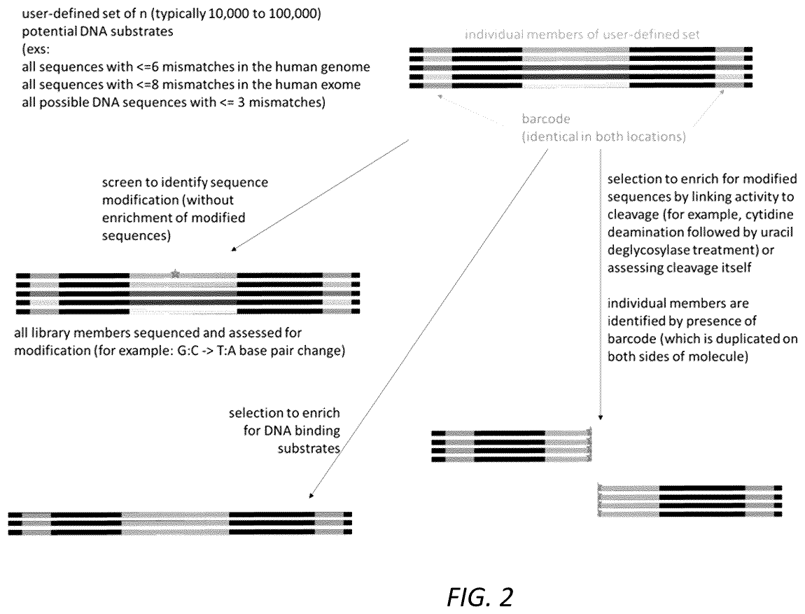

Herein, we provide improved methods (FIG. 2) that enable identification of on- and off-target binding, modification, or cleavage sites of DNA modifying proteins/protein complexes (including but not limited to: dCas9 fused to an effector domain, Cas9-based base editors, or active Cas9 proteins) and that overcomes the disadvantages of both the “genomic DNA” and “random base substitution library” approaches. With this method, a pre-enriched library of linear DNAs consisting of particular user-specified sequences is generated by high-density oligonucleotide synthesis and then interrogated for those sequences that can be bound, modified or cleaved by sequence-specific proteins or protein complexes. Minimally, this method allows for the identification of sequences that are potential substrates for any agent whose modifying action can lead to sequence modification, binding, or cleavage of nucleic acid.

The pre-enriched linear DNA library members are initially synthesized on high-density oligonucleotide arrays as individual single-stranded DNA sequences, each bearing a unique identifier/barcode, which is present/duplicated on both sides of the oligonucleotide (FIG. 2). The synthesized oligos are released from the chip and converted into double-stranded DNA molecules by priming against a common sequence present in all DNA molecules synthesized on the chip. This pooled library is then incubated with a site-specific nuclease, modifying protein, or DNA binding domain of interest and either enriched for cleaved, modified, or bound sequences in a selection format (see examples 1, 3, and 4) or screened for modification (see example 2). The DNA sequences of cleaved sites can then be reconstructed from either of the identical barcodes that originally flanked these sites and that are now separated into two molecules. Synthesized molecules can be specified to represent 1) a set of all potential off-target sequences in a reference genome bearing up to a certain number of mismatches relative to the on-target site (analogous to genomic DNA libraries), 2) a comprehensive set of potential off-target sites bearing up to a certain number of mismatches (analogous to random base substitution libraries), 3) a library of potential off-target sequences present in a set of variant genomes from defined populations (i.e., genomic DNA libraries designed to reflect DNA sequence variants present in a population of individuals), or 4) other relevant defined sets of potential off-target sites (for example, oncogene hotspots or sequences from tumor suppressor genes). This strategy has important advantages for constructing these libraries. For random base substitution libraries, all sequences within a defined number of substitutions can be represented equally and can be easily sampled with current next-generation sequencing methodologies. For genomic or exomic DNA libraries, only the sites that are most likely to be relevant (for example, all potential off-target sites with six or fewer substitutions) are included, which eliminates the noise contributed by the ˜99.998% of sites that are not substrates. Importantly, because this method results in the generation of a double-strand DNA (or RNA) library of an enriched set of potential off-target sites of a DNA (or RNA) binding protein, it can be used to define the specificity and off-target sites of not only nucleases but also other proteins that bind or modify nucleic-acid, including but not limited to, customizable base editing enzymes (Komor et al. Nature 533: 420, 2016, Gaudelli et al. Nature. 551: 464 (2017)), transcriptional activators (Mali et al, Nat Biotech. 31: 833 (2013), Chavez et al. Nat Meth. 12: 326 (2015)), transcriptional repressors (Bikard et al, Nucleic Acids Res, 41: 7429 (2013), Thakore et al. Nat Meth. 12: 1143 (2015)), and epigenome editing enzymes (reviewed in Zentner and Henikoff. Nat Biotech. 33: 606 (2015)).

The ability to define identical barcodes flanking a defined recognition site represents a significant advance over previous in vitro profiling methods (U.S. Pat. Nos. 9,322,006, 9,163,284), because the sequence of the library member is encoded in at least three locations on each individual member of a DNA pool. This redundancy of information is particularly advantageous when seeking to define DNA modifying activity (such as base editing) where the target sequence is modified. The original sequence information can be obtained from the information content contained in a flanking barcode, even if the actual DNA sequence of the library member itself is modified. The redundancy of information in two barcodes and a recognition site also allows for an endonuclease cleavage selection (or paired base modification+cleavage selection) to be performed on potential cleavage sites that are present in a single copy per library member, as opposed to multiple copies (U.S. Pat. Nos. 9,322,006, 9,163,284). Without the present barcoding strategy, sequences of library members that get cleaved within a recognition site cannot be reassembled, since the cut separates in space the two sides of the cut site (above figure, bottom right, blue region).

### Examples

The invention is further described in the following examples, which do not limit the scope of the invention described in the claims.

Target sites used in following examples:

| | | SEQ ID |
|---|---|---|
| target name | Sequence (5′→3′) | NO: |
| EMX1 | GAGTCCGAGCAGAAGAAGAAGGG | 22 |
| RNF2 | GTCATCTTAGTCATTACCTGAGG | 42 |
| FANCF | GGAATCCCTTCTGCAGCACCTGG | 43 |
| HBB | TTGCCCCACAGGGCAGTAACGG | 44 |
| HEK2 (HEK293_2) | GAACACAAAGCATAGACTGCGGG | 45 |
| HEK3 (HEK293_3) | GGCCCAGACTGAGCACGTGATGG | 46 |
| HEK4 (HEK293_4) | GGCACTGCGGCTGGAGGTGGGGG | 47 |
| ABE14 (ABE_14) | GGCTAAAGACCATAGACTGTGGG | 48 |
| ABE16 (ABE_16) | GGGAATAAATCATAGAATCCTGG | 49 |
| ABE18 (ABE_18) | ACACACACACTTAGAATCTGTGG | 50 |
| VEGFA3 (VEGFA_3) | GGTGAGTGAGTGTGTGCGTGTGG | 51 |

### Example 1: DNA Cleavage Selection with SpCas9 and SpCas9-HF1

In this example, a random base substitution library designed for an SpCas9 nuclease programmed with a guide RNA (gRNA) designed against an on-target site in the human EMX1 gene (hereafter referred to as the EMX1 gRNA and EMX1 target site) and a library of potential EMX1 gRNA off-target sites from the human reference genome were selected for cleavage with SpCas9 or SpCas9-HF1.

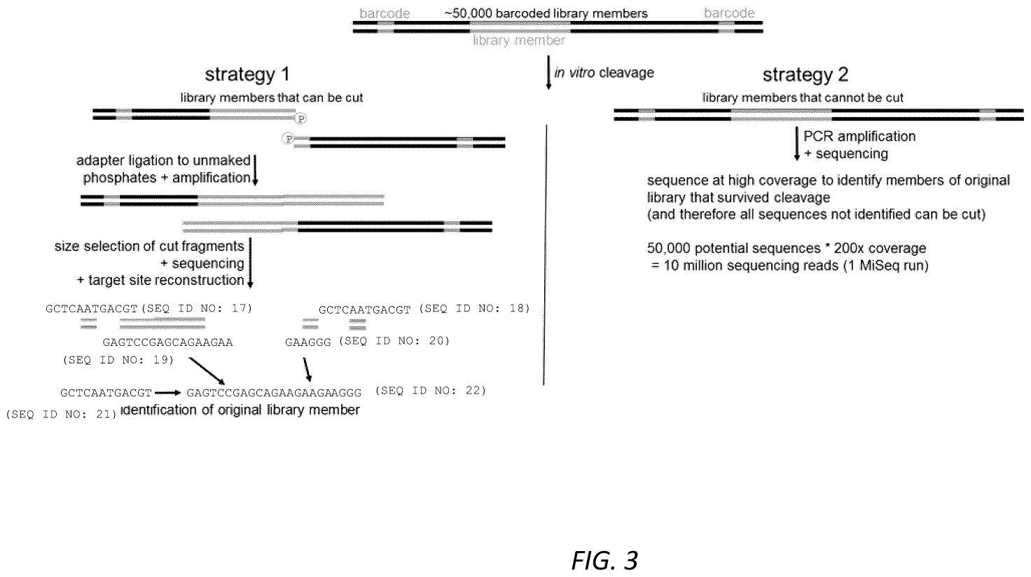

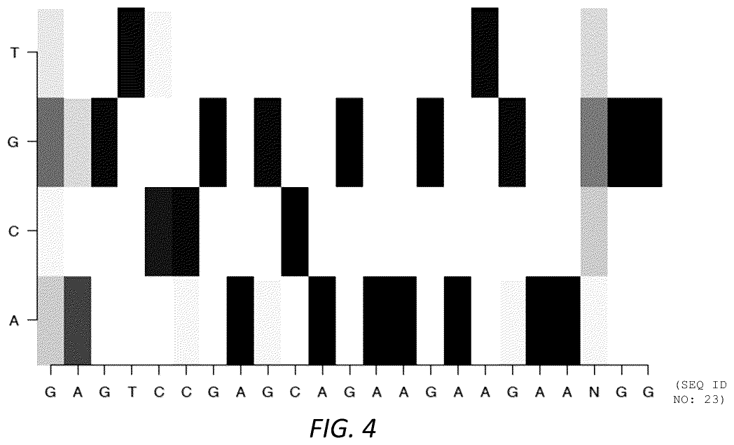

In this example (FIG. 3), both a selection (strategy 1) and a screen (strategy 2) can be employed. In strategy 1, a pooled library of ˜50,000 barcoded library members (either a random base substitution library containing all possible sequences within three mismatches of an EMX1 SpCas9 on-target site or a genomic DNA-inspired library containing all possible sequences from the hg19 human reference genome within six mismatches of an EMX1 SpCas9 on-target site—see Methods for details of libraries.) A selection performed using strategy 1 on the random base substitution library with a 1:1:1 ratio of SpCas9:sgRNA:DNA library (EMX1 target sites) demonstrated enrichment of sequences that could be cleaved (FIG. 4). The positions in the target site are on the horizontal axis (with the on target bases listed below). The possible bases (substitutions or on-target) in the library are indicated on the vertical axis. Data was pooled and summarized in a heatmap, where darker black rectangles indicate a larger proportion of sites in the post-selection library containing the corresponding base from the vertical axis. As a proof of principle, this heatmap agrees with previous studies that demonstrate the N of the NGG PAM sequence is not specified and that specificity at the PAM-distal end of the target site is lower than that of the PAM-proximal end.

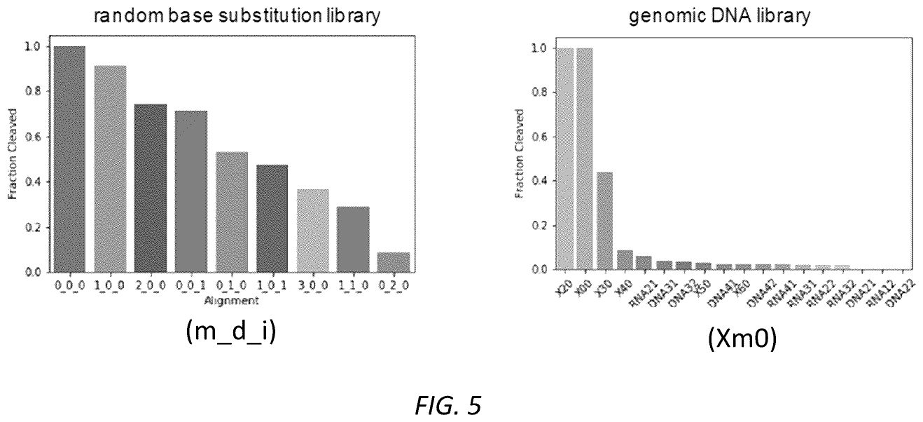

A screen performed using strategy 2 with the random base substitution library yielded similar results (FIG. 5). Substrates with fewer numbers of mutations (indicated by lower m numbers, where (m_d_i)→m=number of mutations, i=number of insertions, d=number of deletions) in the substrate profiling library and in the genome-inspired library where Xm0 denotes m mismatches without any insertions, RNAmd indicates a target site deletion of length d with m mismatches at the remaining base pairs in the site, DNAmi indicates a target site insertion of length I with m mismatches at the remaining base pairs in the site).

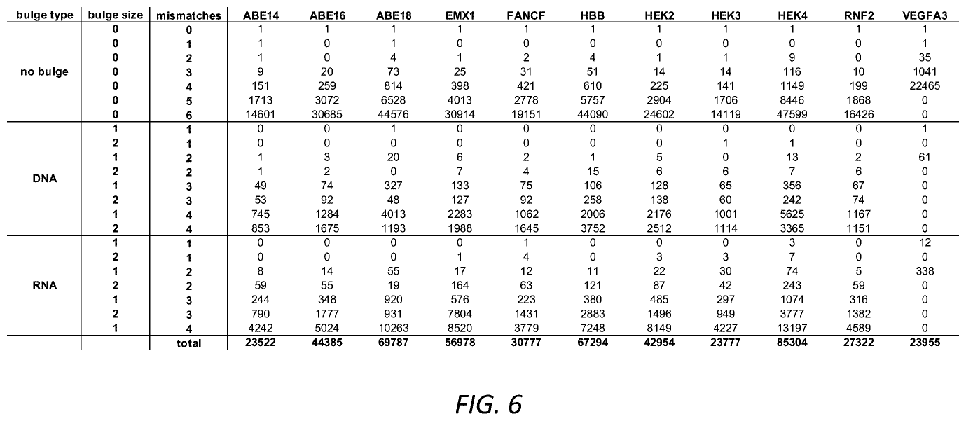

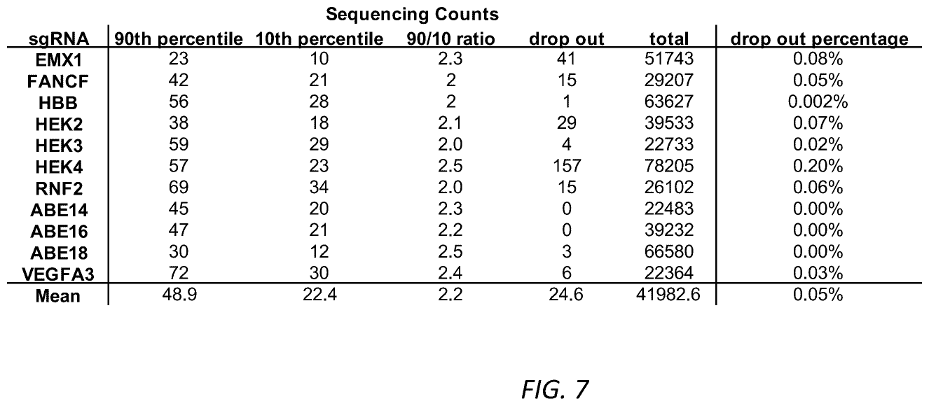

Genomic libraries are generally composed of all potential off-target sites in the hg19 reference human genome that had zero to six mismatches relative to the on-target sequence, up to four mismatches in combination with a DNA bulge of one or two nucleotides, and up to four mismatches with an RNA bulge of one nucleotide, and up to three mismatches with an RNA bulge of two nucleotides (FIG. 6). Sequencing of pre-selection libraries to assess quality metrics (FIG. 7) demonstrated a low dropout rate (0.20% or less) and high uniformity (90/10 ratio>−2). These metrics have not, to our knowledge, been calculated for other specificity methods, so direct comparison is not possible.

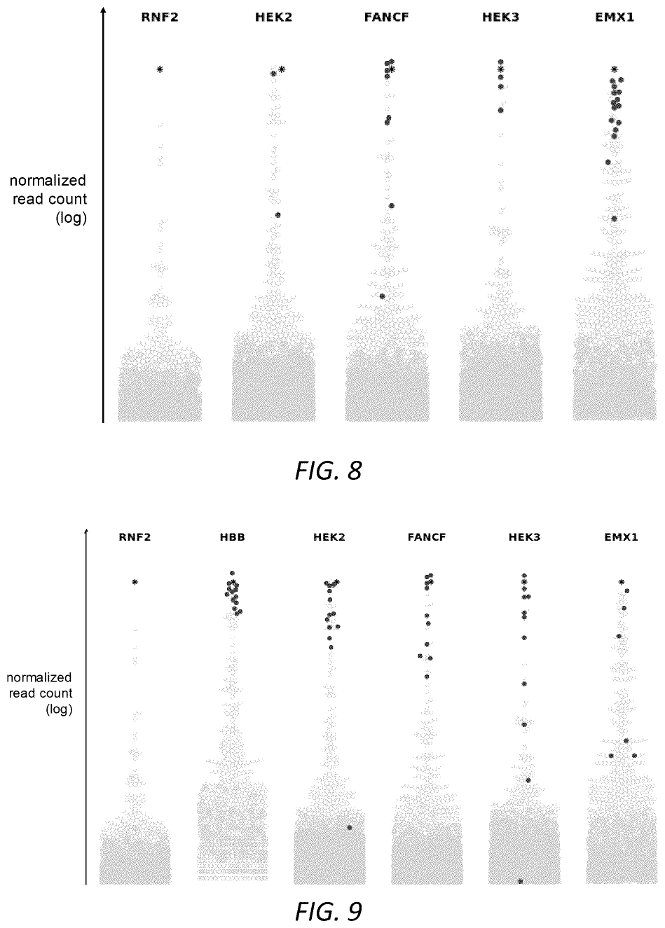

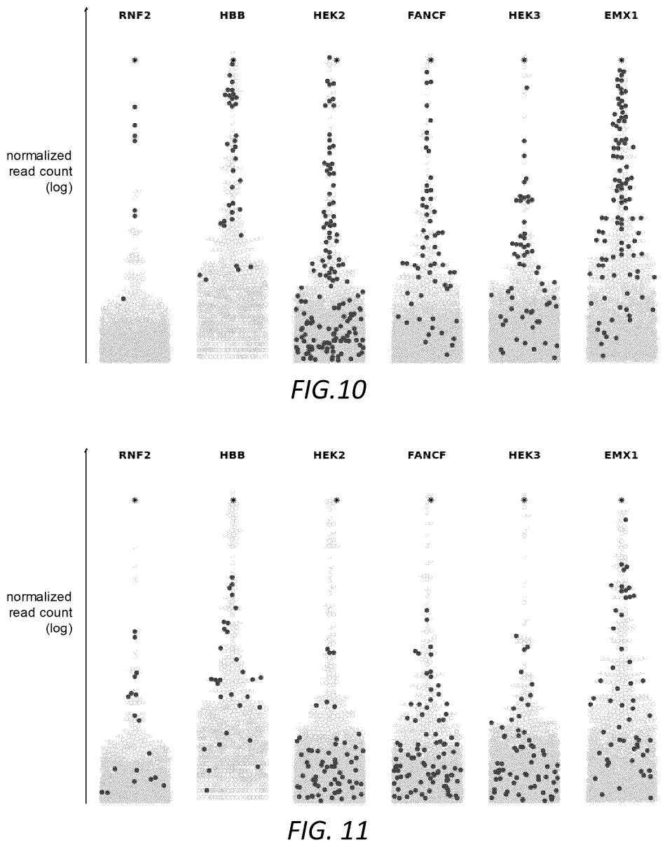

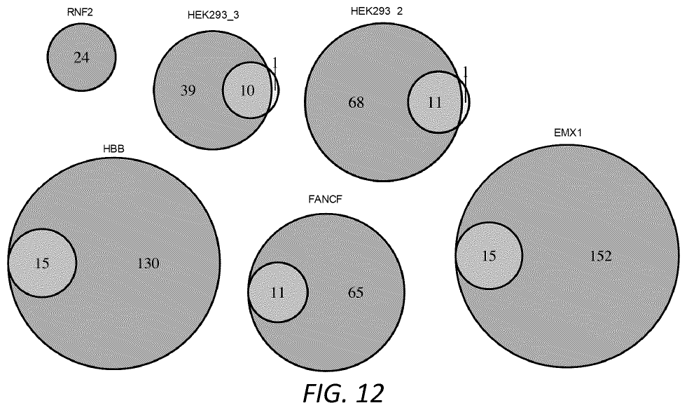

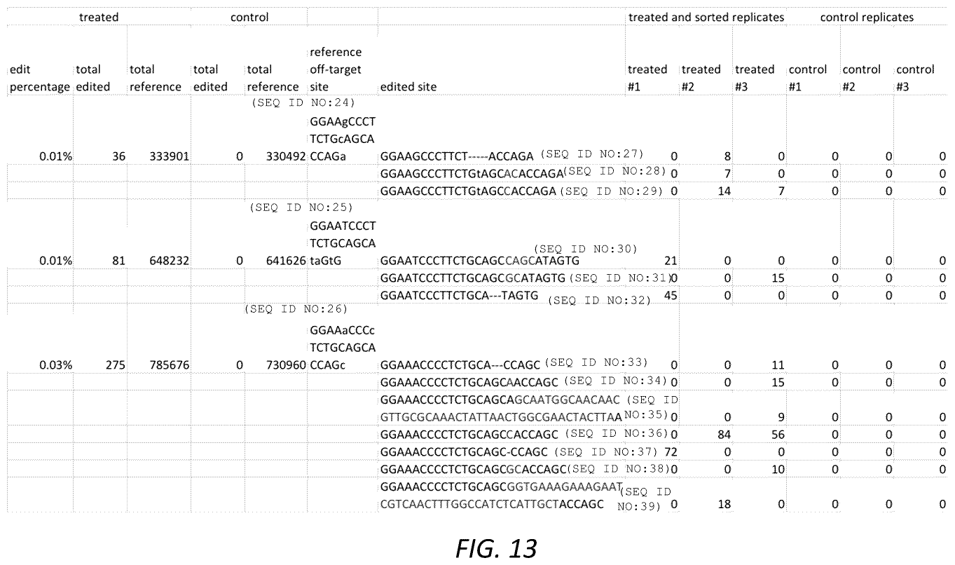

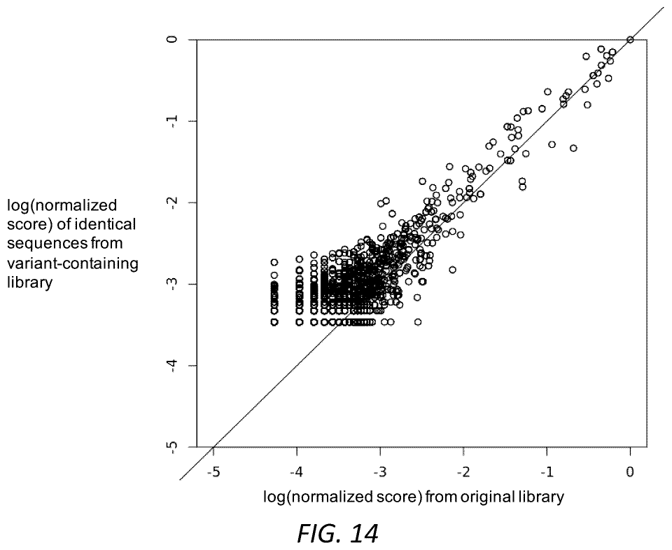

Selections were performed using strategy 1 (referred to as ONE-seq) with genomic DNA-inspired libraries for six non-promiscuous guide RNAs (HBB, RNF2, HEK2, HEK3, FANCF, and EMX1) with relatively few expected off-target sequences. On target sequences (FIG. 8, black stars) were either the most enriched or among the top 3 most enriched library members out of tens of thousands for the six non-promiscuous guide RNAs tested. Summed over the six non-promiscuous guide RNAs, ONE-seq enriched all 163 GUIDE-seq identified off-target sites (FIG. 8, filled circles), with post-selection read counts of ranging from 11% to 120% of the on-target sequences. This method also enriches highly enriched CIRCLE-seq sites (defined here as those with >100 sequence reads, FIG. 9), and appropriately, enriches to a lesser extent CIRCLE-seq sites that are moderately enriched (10-99 reads, FIG. 10), or lowly enriched (1-9 reads, FIG. 11). If a cutoff of 1% of the on-target enrichment in the ONE-seq method described here, ONE-seq identifies 60 out of 62 highly enriched CIRCLE-seq sites (FIG. 12), while CIRCLE-seq fails to identify 478 highly enriched ONE-seq candidates. Of note, the 2 highly enriched CIRCLE-seq sites that are not highly enriched by ONE-seq may be false positives of the CIRCLE-seq method. Validation of ONE-seq sites that are novel and not identified by GUIDE-seq or CIRCLE-seq was demonstrated by sorting HEK293T cells in the top decile of SpCas9: FANCF sgRNA expression (FIG. 13). These results demonstrate that the method described here is at least as sensitive as existing methods and is likely more sensitive.

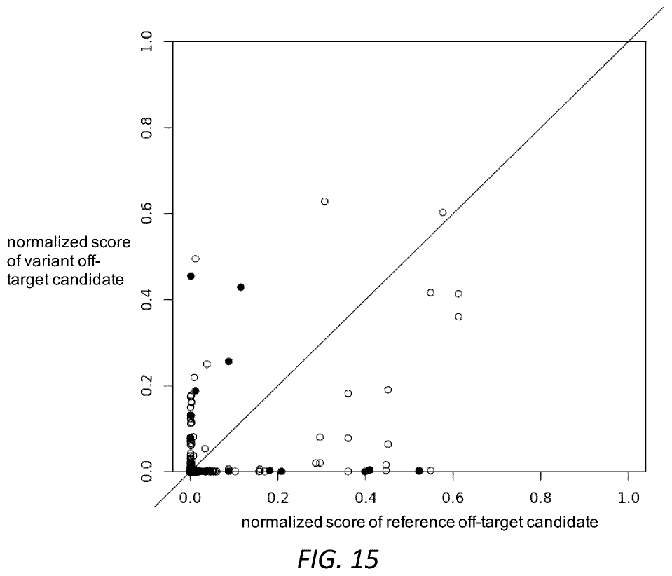

In addition, this method can be generalized to any library/defined set of nucleic acid sequences. For example, using publicly available data from the 1000 genomes project, ONE-seq selections were performed on an EMX1 genomic off-target site library that accounts for naturally occurring sequence variation on a population scale. In this example library, all sequences from the reference hg19 human genome assembly that were in the original EMX1 library (FIG. 6) and contained a SNP in the 1000 genomes database were included. In addition, the SNP containing sequences were also included as additional library members to account for the possibility that individuals may have off-target sequences that are not contained in the reference genome. A ONE-seq cleavage selection performed on this SNP-containing EMX1 library provides reproducible enrichment of off-target candidates that are present in the reference hg19 genome (FIG. 14). ONE-seq cleavage selections on the variant library demonstrate assessment of tens of thousands of variants present in the population for a candidate set of off-target sites of the EMX1 guide RNA, identifying several (FIG. 15, black circles) that are differentially enriched.

### Example 2: Base Editor Screen with BE1

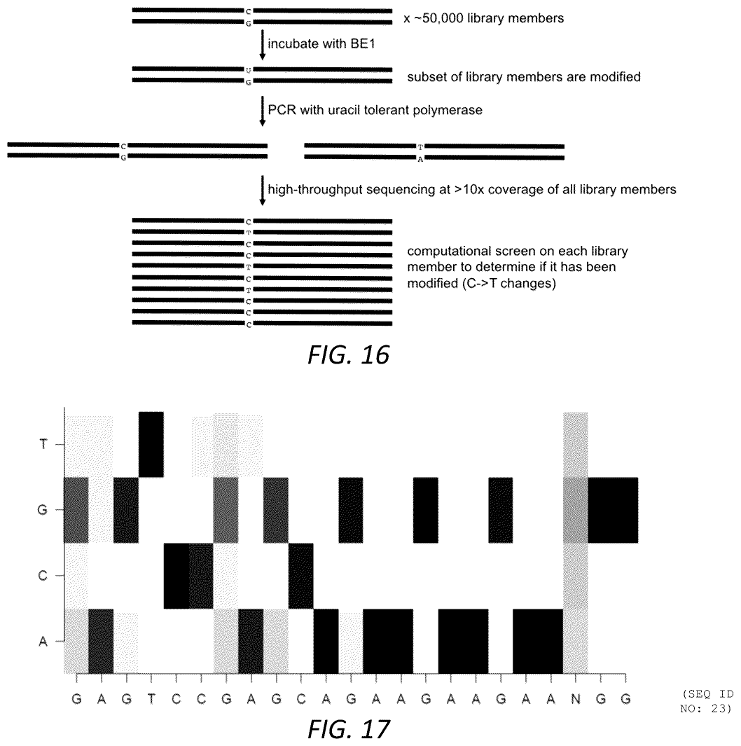

In this example (FIG. 16), a screening strategy is used to identify base modification created by the BEI enzyme (Komor et al. Nature 533: 420, 2016), which canonically creates C→U changes in a defined window of DNA.

A base editor screen following the protocol above with BEI was applied to an EMX1 target site and the substrate profiling library yielded enrichment of an expected profile of tolerated off-target sites (FIG. 17).

### Example 3: Base Editor Selection with BE3

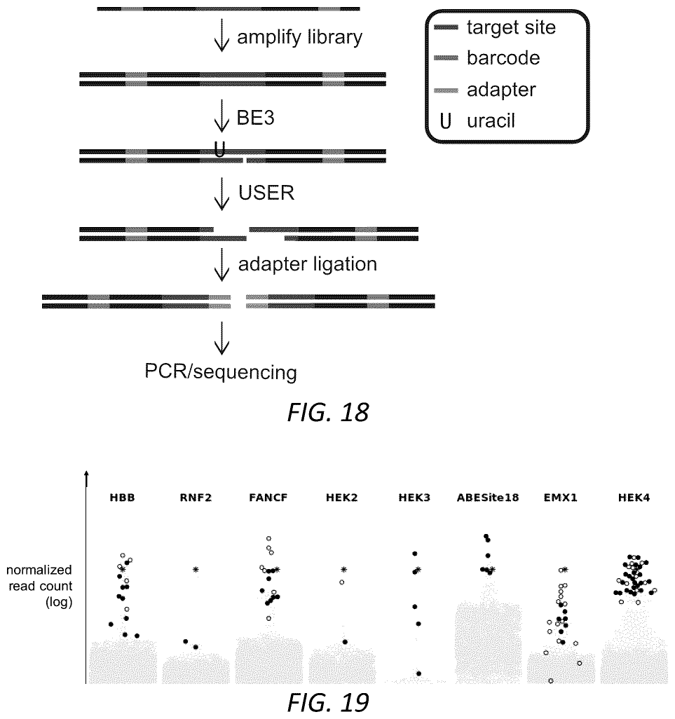

In this example (FIG. 18), a selection strategy was used to enrich for sites that are modified by the BE3 enzyme. Library members that can be recognized by the BE3 enzyme (Komor et al. Nature 533: 420, 2016) should exhibit both C→U modification and a nick on the opposite strand. The USER enzyme (NEB) was used to achieve double stranded cleavage of library members that are BE3 substrates by replacing dU nucleotides with a nick. Resulting modified library members will therefore contain two nicks with 5′ phosphates on opposite strands and are incubated with a DNA polymerase that can blunt these DNA overhangs (ex: T4 DNA polymerase or Phusion DNA polymerase). The resulting phosphorylated blunt ends are captured with double stranded DNA adapters prior to amplification/selection using one primer specific to the adapter and one primer specific to the library backbone (as in Example 1). Additional selection stringency can be obtained by performing size selection for smaller, cut fragments before or after amplification.

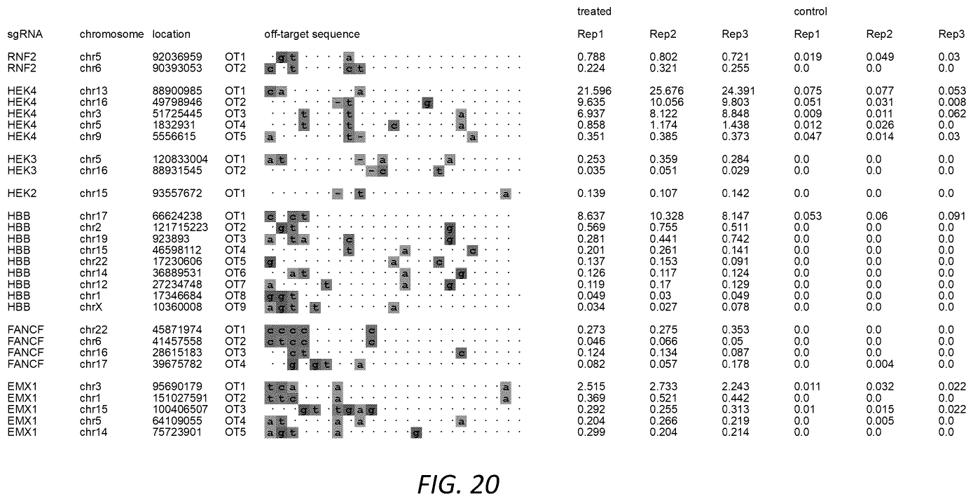

Using this approach, we examined BE3 targeting with genomic DNA-inspired libraries for eight target sites, including all seven BE3 targets tested previously by Digenome-seq (Kim et al. Nat. Biotech. 35:475, 2017). ONE-seq selection results revealed enrichment of the intended target sites to the top 13 of tens of thousands of library members for all eight selections (FIG. 19, black stars). For three out of the eight selections, the intended target site was the most enriched site. All 42 previously validated off-target sites by Digenome-seq were present in the enriched, post-selection libraries (FIG. 19, open black circles), and 40 out of the 42 were among the top 61 sites for each selection. To further validate our ONE-seq results, we amplified and sequenced from human HEK293T cells approximately 20-40 highly-ranking sites from each selection. Our results demonstrated 28 validated BE3 off-target sites that were not identified as candidates by Digenome-seq (FIG. 19, solid black circles). Six of the 28 new sites had edit percentages greater than 1% in cells (FIG. 20), with a high of 23.9%, suggesting a higher level of sensitivity of ONE-seq for detecting not only weak off-target sites but also high frequency off-target sites.

### Example 4: Base Editor Selection with ABE

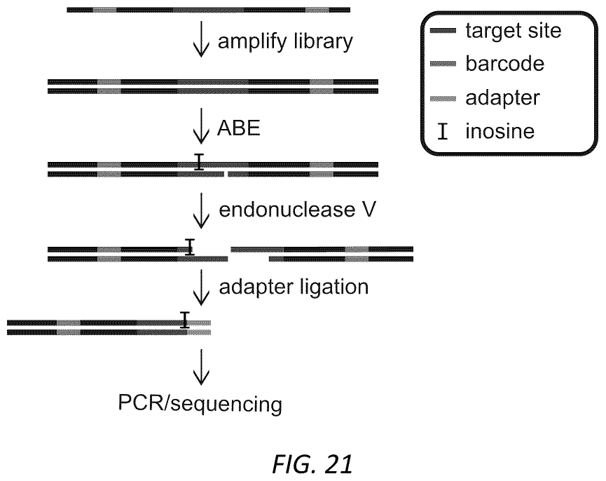

In this example (FIG. 21), we performed a selection using Adenine base editor (ABE; Gaudelli et al. Nature. 551: 464 (2017)) with the EMX1 gRNA and a base substitution profiling library and the genomic DNA library. ABEs are sgRNA-guided Cas9 nickases fused to a protein domain that can catalyze the conversion of deoxyadenosine to deoxyinosine. In this example, double strand cleavage of the pre-selection library is accomplished in two steps (FIG. 21). First, incubation with ABE enzyme and guide RNA leads to nick formation of the strand of a recognized library member that hybridizes to the guide RNA. Second, subsequent incubation with Endonuclease V, an enzyme that creates a nick to the 3′ of deoxyinosines in library members that could result from ABE activity, leads to nick formation on the non-hybridized DNA strand, leading to a double strand break with an overhang. Subsequent fill-in of the double strand break with a DNA polymerase leads to the formation of blunt ends, which can be selected for as described for Cas9 nucleases in Example 1.

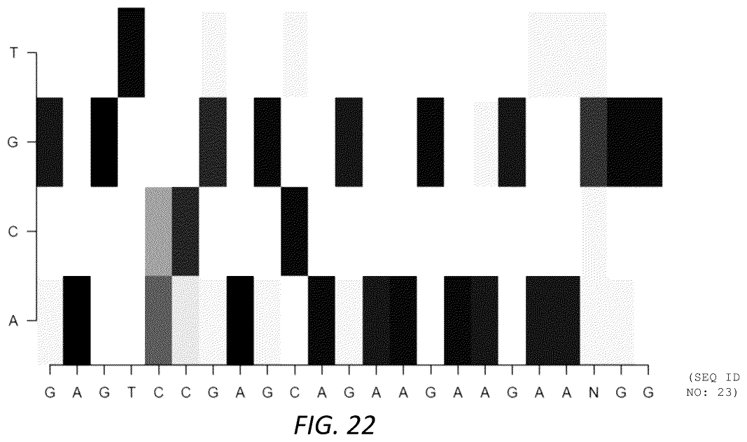

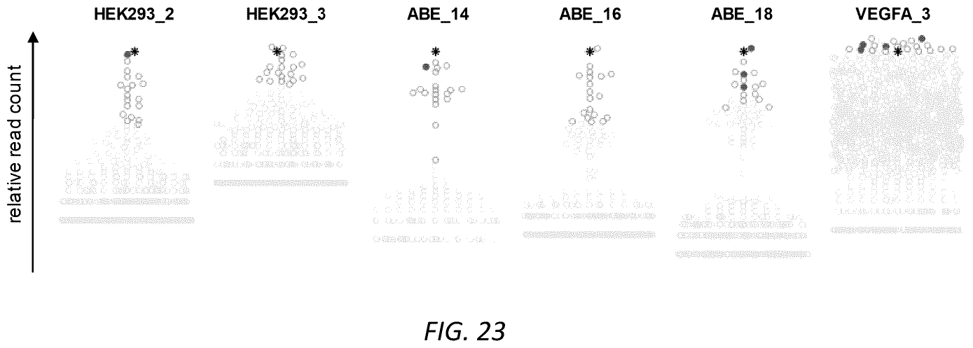

Selection of the base substitution library demonstrates enrichment of substrates with an NGG. In addition, as expected, this experiment (FIG. 22) demonstrates enrichment of substrates with an A at position five of the target site (where 1 is the base pair most distal from the PAM), reflecting a preference of ABE for modification of an A more distal to the PAM than is present in the canonical EMX1 target site. Of note, of the 100 most abundant sequences in the post-selection library, 95 had an A at position 5. These results demonstrate that our strategy works to enrich and identify off-target sites of the ABE.

TABLE 1
Enrichment of sequences with an A in position 5
in the ABE selection.

| First five | Number of times observed |
|---|---|
| nucleotides | out of the top 100 most |
| of post-selection | enriched post-selection |
| library member | library members |
| GAGTA | 83 |
| AAGTA | 12 |
| GAGTC (canonical first | 3 |
| five nucleotides) | |
| GAAGT | 1 |
| GGAGT | 1 |

We have also performed the above selection on the EMX1 genomic DNA library (Table 2), which demonstrates enrichment of the EMX1 on-target site (highlighted; 964 most abundant post-selection library sequence) and the EMX1 off-target site with the highest off-target recognition (bold and asterisk; 9^{th} most abundant post-selection).

TABLE 2
Top 96 most-enriched sites in the post-selection
library for an ABE selection on a genomic DNA
library of potential EMX1 off-target sites.

| | | | SEQ |
|---|---|---|---|
| chromo- | | | ID |
| some | location | target | NO: |
| chr4 | 33321459 | GTACAGGAGCAGGAGAAGAATGG | 52 |
| chr17 | 72740376 | CAAACGGAGCAGAAGAAGAAAGG | 53 |
| chr10 | 58848711 | GAGCACGAGCAAGAGAAGAAGGG | 54 |
| chr10 | 128080178 | GAGTACAAGCAGATGAAAAACGG | 55 |
| chr6 | 99699155 | GAGTTAGAGCAGAGGAAGAGAGG | 56 |
| chr7 | 141972555 | AAGTCCGGGCAAAAGAGGAAAGG | 57 |
| chr19 | 24250496 | GAGTCCAAGCAGTAGAGGAAGGG | 58 |
| chr11 | 111680799 | CAGTAGTGAGCAGAAGAAGATAGG | 59 |
| chr5 | 45359060* | GAGTTAGAGCAGAAGAAGAAAGG | 60 |
| chr7 | 17446431 | GTCCAAGAGCAGGAGAAGAAGGG | 61 |
| chr12 | 106646073 | AAGTCCATGCAGAAGAGGAAGGG | 62 |
| chr15 | 22366604 | GGAGTAGAGCAGAGGAAGAAGGG | 63 |
| chr10 | 109561613 | GGAACTGAGCAAAAGAAGATAGG | 64 |
| chr11 | 62365266 | GAATCCAAGCAGAAGAAGAGAAG | 65 |
| chr2 | 21489994 | GCGACAGAGCAGAAGAAGAAGGG | 66 |
| chr1 | 234492858 | GAAGTAGAGCAGAAGAAGAAGCG | 67 |
| chr2 | 218378101 | GAGTCTAAGCAGGAGAATAAAGG | 68 |
| chr18 | 32722283 | TGTCCAGAGCAGATGAAGAATGG | 69 |
| chr22 | 22762518 | GAACATGAGCAGAAGAAGAGGAG | 70 |
| chr11 | 34538379 | AGGCCAGAGCAAAAGAAGAGAGG | 71 |
| chr11 | 106142352 | GTACAAGAGCAGGAGAAGAAGGG | 72 |
| chr15 | 91761953 | GAGTCAGGGCAGAAGAAGAAAAT | 73 |
| chr4 | 87256685 | GAGTAAGAGAAGAAGAAGAAGGG | 74 |
| chr4 | 21141327 | AAGCCCGAGCAGAAGAAGTTGAG | 75 |
| chr8 | 128801241 | GAGTCCTAGCAGGAGAAGAAGAG | 76 |
| chr7 | 106584579 | GAGGGGAGCAAAAGAAGGAGGG | 77 |
| chr1 | 117139004 | CAGGGAGAGCAAAAGAAGAGAGG | 78 |
| chr1 | 231750724 | GAGTCAGAGCAAAAGAAGTAGTG | 79 |
| chr15 | 44109746 | GAGTCTAAGCAGAAGAAGAAGAG | 80 |
| chr21 | 23586410 | CAGGGAGAAGAAGAAGAAGGG | 81 |
| chr7 | 2127682 | GAGTTAGAGAAGAAGAAGACTGG | 82 |
| chr10 | 98718174 | ACAATCGAGCAGCAGAAGAATGG | 83 |
| chr1 | 221020698 | GAGTAGGAGCAGATGAAGAGAGG | 84 |
| chr9 | 115729750 | CAGTATGAGCAAAAGAAGAAAGA | 85 |
| chr11 | 102753237 | GAGTCCATACAGAGGAAGAAAAG | 86 |
| chr1 | 48581991 | GAATGAGCAAAAGAAGAAAGC | 87 |
| chr12 | 73504668 | GAGTTAGAGCAGAAAAAAAATGG | 88 |
| chr1 | 184236226 | AATACAGAGCAGAAGAAGAATGG | 89 |
| chr11 | 119322554 | TAGTGAGCAGAAGAAGAGAGA | 90 |
| chr1 | 151027591 | TTCTCCAAGCAGAAGAAGAAGAG | 91 |
| chr11 | 68772640 | GAGTCCATACAGGAGAAGAAAGA | 92 |
| chr2 | 9821536 | AGGTGGGAGCAGAAGAAGAAGGG | 93 |
| chr2 | 54284994 | AAGGCAGAGCAGAGGAAGAGAGG | 94 |
| chr1 | 99102020 | GAGGCACAAGCAAAAGAAGAAAAG | 95 |
| chr19 | 1438808 | GAAGTAGAGCAGAAGAAGAAGCG | 96 |
| chr2 | 73160981 | GAGTCCGAGCAGAAGAAGAAGGG | 22 |

The two sequences highlighted are the most active cleavage off-target site (chr5: 45359060), asterisked, and the on-target site (chr2: 73160981). It is expected for the off-target site to be more enriched in the selection due to presence of an A in a more favorable position in the editing window.

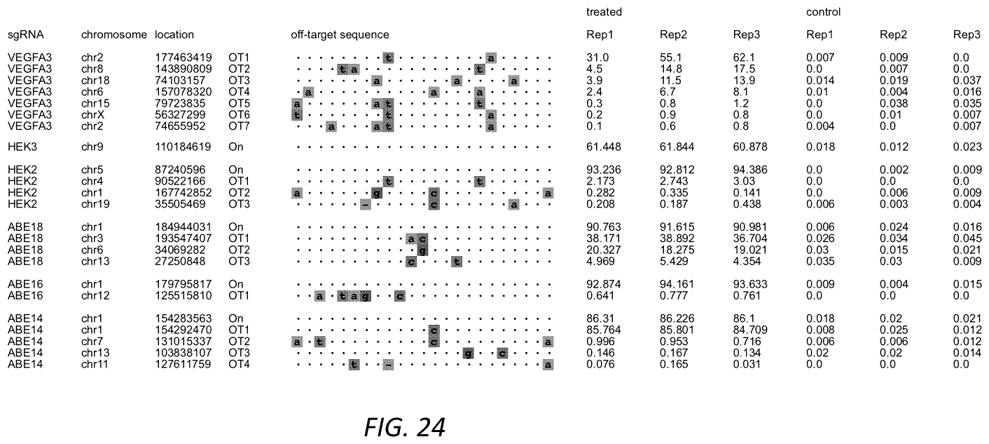

We have additionally performed the above selection on genomic DNA libraries designed to identify off-target sequences of six guide RNAs (FIG. 23). Application of the modified ONE-seq selection protocol to six ABE targets revealed enrichment of the intended, on-target site to the top 3 of the post-selection libraries for the five non-promiscuous guides tested (HEK4 is a known promiscuous guide RNA). Validation by amplicon sequencing of DNA from human HEK293T cells individually transfected with the appropriate ABE7.10:sgRNA pairs of top candidate sites (approximately 20 each from each selection) identified 12 total confirmed cellular off-target sites across six target sites. This set includes the three validated off-target sites identified for the two guide RNAs that were tested by both ONE-seq and EndoV-seq (Liang et al. Nature Communications. 10: 67 (2019)) or Digenome-seq (Kim et al. Nature Biotechnology. 37: 430 (2019)) as well as nine newly validated off-target sites that were not identified as potential candidates by either of those methods. Nine of the 12 sites either had off-target modification rates below one percent or only showed evidence of a single nucleotide substitution, either of which could be caused by sequencing error despite stringent quality filtering of sequencing reads (all positions in paired reads must have quality score >Phred 30) and triplicate validation. To improve our confidence that these sites are bona fide off-target sites, we performed a second round of validation experiments with cells transfected with a plasmid expressing both ABEmax, a codon-optimized version of ABE7.10, and GFP and sorted the cells to enrich for the top decile of GFP, and therefore ABE, expression (FIG. 24). Genomic DNA extraction was performed immediately after sorting, without further expansion. In the sorted validation set, on target modification frequencies ranged from 61%-94%, compared to 31%-56% in the unsorted validation set. All 12 of the off-targets from the unsorted validation set were modified at higher frequencies in the sorted validation set, confirming that they are bona fide off-targets, and five additional off-target sites were identified at modification frequencies of less than one percent. One ABE_14 off-target site, containing a single mismatch relative to the on-target site, was modified in 85% of the DNA in the sorted validation (18% in the unsorted validation), suggesting that some ABE off-target sites can be modified at high frequencies.

### Example 5: Base Editor Selections with ABE or BE3 Using an Enzyme that Creates a Double-Strand Break at Positions that have been Modified

In this example, modified library members containing a deoxyinosine could be made to have blunt, double-stranded ends through the action of the TkoEndoMS protein (Ishino et al, Nucleic Acids Res. 44: 2977 (2016)). TkoEndoMS can be used to create a doublestrand breaks at the dI:dT base pairs that result from dA→dI editing by ABE. DNA with a double strand break is then subject to the same downstream steps as in Example 1, with ligation of adapters to phosphorylated, blunt ended DNA if a base editing enzyme without nicking activity is used. If a base editing enzyme with nicking activity is used, end polishing with a blunt-end creating DNA polymerase (such as T4 or Phusion), such as in Examples 4 and 5, is used to allow for enrichment of both sides of a cut library member.

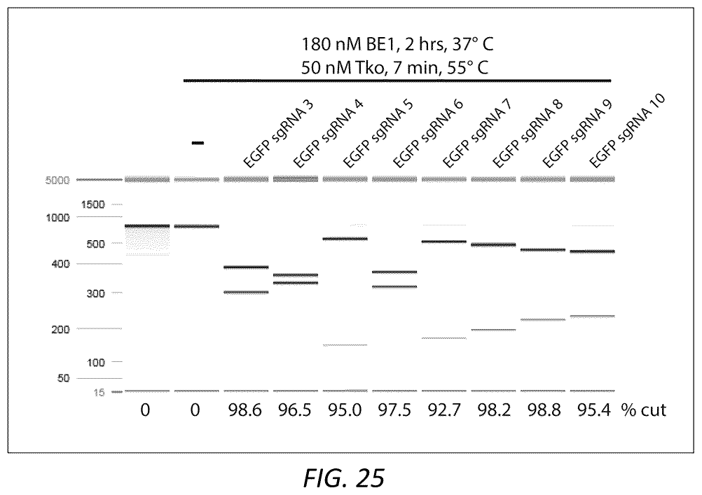

We have demonstrated that TkoEndoMS can also create double-strand breaks at dG:dU mismatched base pairs that result from dC→dU editing (in this example by BE1), demonstrating its additional applicability to BE1, BE3 and other enzymes that cause dC→dU changes after DNA binding (see U.S. Ser. No. 62/571,222 and FIG. 25). This strongly suggests that we can use TkoEndoMS on our synthesized DNA site libraries to identify off-target base edits caused by the various base editors that induce dC to dU edits, regardless of whether a Cas9-induced nick is also present.

### Example 6: Enrichment of DNA Binding Sites by Pulldown

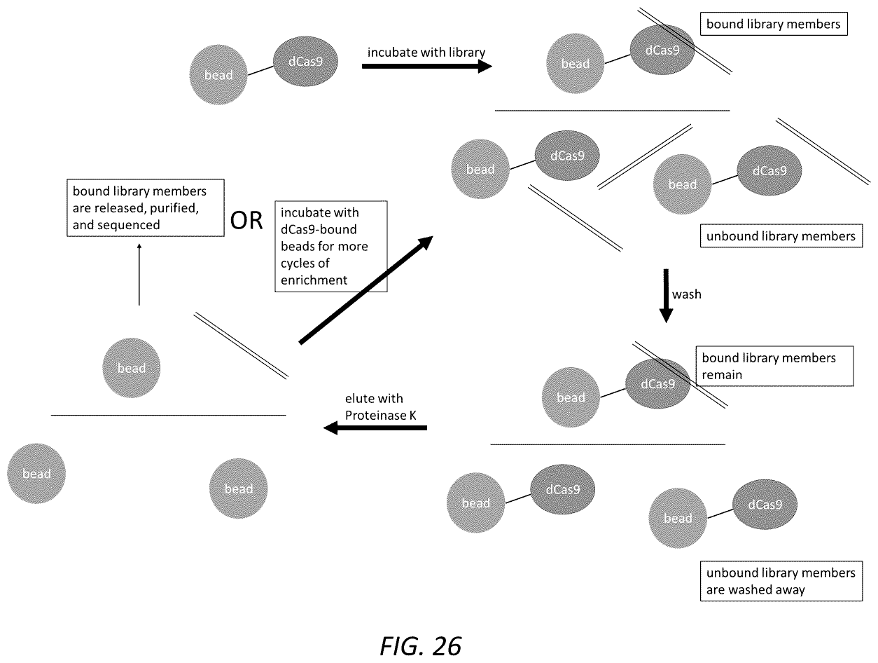

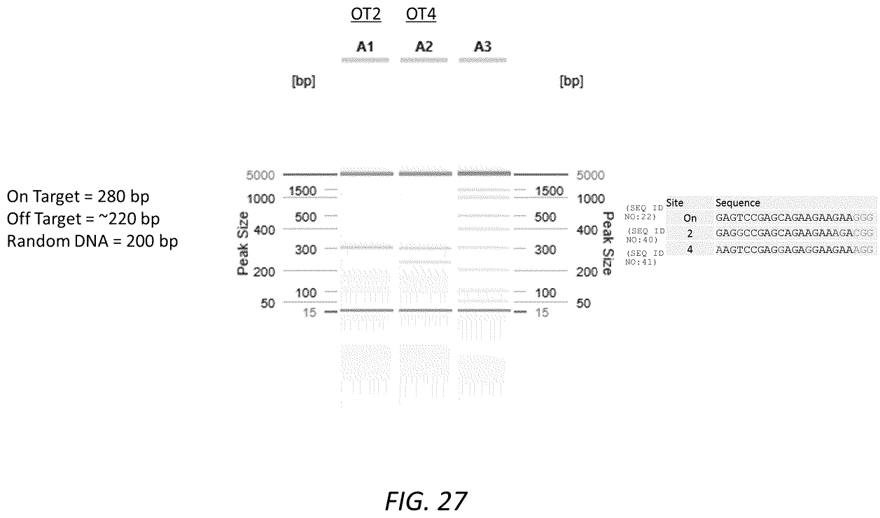

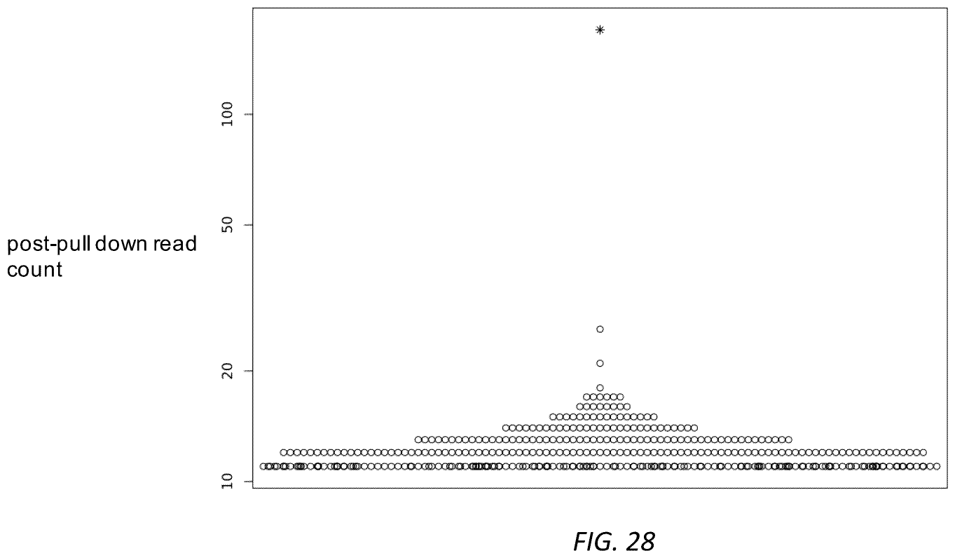

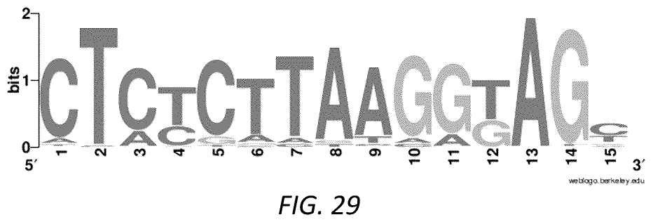

SELEX (selective evolutions of ligands by exponential enrichment) has been used to define the DNA-binding specificity of DNA-binding domains (originally by Oliphant et al., Mol Cell Biol. 9: 2944, 1989). In the SELEX method, libraries of randomized DNA sequences are subjected to multiple rounds of pulldown and enrichment with an immobilized DNA binding domain of interest to identify the sequences in the initial pool that can bind to a DNA of interest. The SELEX method has been applied to the zinc finger and TALE moieties of ZFNs (Perez et al., Nat Biotech. 26: 808 (2008)) and TALENs (Miller et al. Nat Biotech. 29: 143 (2011)), however, there are no reports of SELEX studies on Cas9 proteins. We speculated that SELEX studies on Cas9 proteins are difficult due to the need to selectively enrich a 22 base pair target site from a large library, which would have to contain >10¹³ unique molecules, or at minimum 10¹² molecules, corresponding to a 20 base pair target site, if an NGG PAM is fixed. In this example, we took advantage of pre-enriching our pre-selection libraries for sites that are most likely to be bound by a given Cas9:sgRNA complex (or other DNA-binding domain with predictable binding motifs). We assessed Cas9 DNA binding preferences and specificity by performing sequential rounds of DNA pull down experiments on the pre-enriched libraries (FIG. 26). This was achieved by tethering inactivated Cas9 (dCas9) to magnetic beads. To chemically bind dCas9 to magnetic beads we employed Cas9 protein harboring a so called SNAP-tag. Proteins with a SNAP-tag can be covalently bound to a benzylguanine-carrying substrate molecule, such as a magnetic bead. We envision incubating either type of oligonucleotide library with bead-bound SNAP-tagged dCas9 and enriching for DNA substrates with a high binding affinity to Cas9 by magnetic bead capture of bound sequences and washing away unbound sequences. This process could be repeated in multiple cycles by amplifying eluted library members and using the resulting enriched DNA library as a starting library for bead-based selection. Using this method, with a single cycle, we have demonstrated conditions that could lead to selective pulldown with a dCas9: EMX1 sgRNA of the on target site compared to an off target site (FIG. 27). Furthermore, pulldown of a FANCF genomic DNA-inspired library, leads to maximal enrichment of the on-target site relative to other sites (FIG. 28). The on target site Detailed knowledge of Cas9 binding will be especially valuable to mechanistically study improved properties of genetically engineered Cas9 variants, such as high fidelity Cas9. Importantly, off-target patterns of Cas9 fusion proteins (or fusion proteins with other DNA binding domains) with limited interdependence of effector domain and DNA binding domain might be mainly defined by the DNA binding properties of the fusion protein. Performing DNA pulldown experiments on pre-enriched oligonucleotide libraries might therefore contribute to our understanding of fusion protein off-target distributions. Furthermore, by conducting DNA binding studies of Cas9 (or other DNA binding protein domains) on a library with limited complexity, high quality binding data with little background noise can be obtained that could be subsequently used to extrapolate and predict binding of more complex libraries, such as the genome of a cell.

### Example 7: Homing Endonuclease Selections

Homing endonucleases, such as I-PpoI, represent a group of naturally occurring nucleases that have longer base recognition motifs than the majority of restriction enzymes. Though homing endonucleases (also called meganucleases) do not have specificities that can be easily reprogrammed, if they target a genomic sequence of interest, they could be of research, commercial, or clinical use. Here, we show that we could adapt our in vitro selection to analyze the specificity profile of the I-PpoI homing endonuclease. We created an unbiased library of potential I-PpoI off-targets including all sites with up to 3 mismatches and single DNA/RNA bulges. The I-PpoI library contained 15533 members. I-PpoI selections enriched 501 of the 15533 library members (Table 3) while the intended, on-target site was ranked close to the top of the selection (28 out of 15533). Sequences with one mismatch or one insertion were the most enriched library members. Analysis of mismatch positions among top scoring I-PpoI off-target candidates revealed that certain positions within the recognition motif were more important for I-PpoI cleavage than others (FIG. 29). Especially positions 2, 13 and 14 seemed to be highly conserved and most important for I-PpoI mediated DNA cleavage. The adaptation of the in vitro selections to homing endonucleases demonstrates that the selections are broadly usable to analyze the off-target profiles of a variety of nucleases, including those that get cut to reveal sticky ends (like I-PpoI and Cas12a). I-PpoI leaves a 4 bp 3′-overhang, a DNA end configuration that is known to decrease the efficiency of off-target detection by existing methods such as GUIDE-seq or CIRCLE-seq. We therefore demonstrate that the in vitro selections can be used to analyze nucleases inducing staggered DNA breaks.

TABLE 3
Top 30 most-enriched sites in the post-selection
library for I-PpoI on a unbiased DNA library.

| | | | Found | |
|---|---|---|---|---|
| Align- | | | seqs_ | Found seqs_ |
| ment | target | # | cleaved | cleaved_rmv |
| 1_0_1 | CTATCTTAAGGTAGTC | 97. | 1507 | 1459 |
| 1_0_1 | ACTCTCTTAAGGTAGC | 98. | 1329 | 1294 |
| 1_0_1 | CTATCTTAAGGTAGCC | 99. | 1264 | 1235 |
| 3_0_0 | CTACCTTAAGGTAGT | 100. | 1100 | 1071 |
| 3_0_0 | CTACCTTAAGGGAGC | 101. | 1017 | 989 |
| 2_0_0 | CTATCTTAAGGGAGC | 102. | 967 | 951 |
| 2_0_0 | CTCCCTTAAGGGAGC | 103. | 960 | 923 |
| 1_0_1 | CTATCTTAAGGTAGGC | 104. | 947 | 919 |
| 1_0_1 | CTCTCTTAAGGGAGCC | 105. | 920 | 896 |
| 1_0_1 | CTCTCTTAAGGTAGCT | 106. | 913 | 883 |
| 2_0_0 | CTCCCTTAAGGTAGT | 107. | 885 | 866 |
| 0_0_1 | CTCTCTTAAGGTAGTC | 108. | 865 | 842 |
| 1_0_1 | CTCTCTTAAGATAGCC | 109. | 858 | 836 |
| 2_0_0 | CTACCTTAAGGTAGC | 110. | 829 | 799 |
| 1_0_1 | CTCCCTTAAGGTAGTC | 111. | 781 | 765 |
| 1_0_1 | CTCTCATAAGGTAGTC | 112. | 744 | 724 |
| 1_0_1 | CTCTCATAAGGTAGCC | 113. | 744 | 722 |
| 1_0_1 | CTCTGTTAAGGTAGTC | 114. | 729 | 710 |
| 3_0_0 | CTCCCTTAAGAGAGC | 115. | 732 | 702 |
| 1_0_1 | CTCCCTTAAGGTAGCC | 116. | 713 | 694 |
| 1_0_1 | CTCCCTTAAGGTAGAC | 117. | 709 | 689 |
| 1_0_0 | CTCTCTTAAGGTAGT | 118. | 679 | 670 |
| 2_0_0 | CTATCTTAAGGTAGT | 119. | 687 | 670 |
| 1_0_0 | CTCTCTTAAGGGAGC | 120. | 673 | 653 |
| 3_0_0 | CTATCTTAAGGGAGT | 121. | 651 | 639 |
| 1_0_1 | CTCTGTTAAGGTAGCC | 122. | 650 | 636 |
| 0_0_1 | CTCTCTTAAGGTAGGC | 123. | 650 | 633 |
| 0_0_0 | CTCTCTTAAGGTAGC | 124. | 643 | 628 |
| 1_0_0 | CTATCTTAAGGTAGC | 125. | 629 | 620 |
| 2_0_0 | CTCTCTTAAGAGAGC | 126. | 634 | 620 |

#, SEQ ID NO:
Mostly closely matched off-target candidates were enriched to the top of the selection. However, the selections demonstrated that I-PpoI off-target candidates are exitent in abundance

### Methods: Library Generation

Oligonucleotide library synthesis on high density chip arrays were purchased from Agilent.

### Substrate Profiling Library:

1) An oligonucleotide backbone was developed that had 50% GC content and no potential canonical PAM sequences (NGG for S.pyogenes Cas9).
2) 13-14 base pair barcodes were generated that were at least two substitutions away from all other barcodes, were 40-60% GC, and did not contain any canonical PAM sequences for the minimally unbiased libraries:
3) potential off-target sites were generated for all possible combinations of substitutions, insertions, and deletions for an SpCas9 target site (this can be variable):

| | single base | single base |
|---|---|---|
| | pair | pair |
| substitutions | deletions | insertions |
| <=3 | 0 | 0 |
| <=1 | 1 | 0 |
| 0 | 2 | 0 |
| <=1 | 0 | 1 |

4) barcodes/potential off-target sites for all i off target sites (I˜50,000) were combined into the backbone:

(SEQ ID NO: 127)
GACGTTCTCACAGCAATTCGTACAGTCGACGTCGATTCGTGCT(bar-
codeᵢ)TTTGACATTCTGCAATTGCACACAGCGT(potential_off_
target_siteᵢ)TGCAGACTGTAAGTATGTATGCTTCGCGCAGTGCGA
CTTCGCAGCGCATCACTTCA(barcodeᵢ)AGTAGCTGCGAGTCTTACA
GCATTGC

### Genome-Inspired Library:

1) Potential off-target sites were generated with CasOffFinder according to the table below (these parameters can vary) and 20-113 bp (this can be variable) of genomic flanking sequence was added

| | single base | single base |
|---|---|---|
| | pair | pair |
| substitutions | deletions | insertions |
| <=6 | 0 | 0 |
| <=4 | <=2 | 0 |
| <=3 | 0 | <=2 |
| 4 | 0 | 1 |

For an EMX1 site, here is an example of the number of sequences present given the above parameters.

| | insertion | deletion | |
|---|---|---|---|
| | (DNA bulge) | (RNA bulge | # of |
| mismatches | length | length) | sequences |
| 0 | 0 | 0 | 1 |
| 2 | 0 | 0 | 1 |
| 3 | 0 | 0 | 25 |
| 4 | 0 | 0 | 378 |
| 5 | 0 | 0 | 3903 |
| 6 | 0 | 0 | 30213 |
| 1 | 0 | 2 | 1 |
| 2 | 1 | 0 | 6 |
| 2 | 2 | 0 | 7 |
| 2 | 0 | 1 | 17 |
| 2 | 0 | 2 | 161 |
| 3 | 1 | 0 | 130 |
| 3 | 2 | 0 | 126 |
| 3 | 0 | 1 | 566 |
| 3 | 0 | 2 | 7579 |
| 4 | 1 | 0 | 2214 |
| 4 | 2 | 0 | 1942 |
| 4 | 0 | 1 | 8279 |
| | | Total | 55549 |

2) barcodes/potential off-target sites for all i off target sites (i˜50,000) were combined into the backbone as for the minimally unbiased library with maximal genomic flanking context:
GACGTTCTCACAGCAATTCGT(barcodeᵢ)(flanking genomic contexti)(potential_off_target_sitei)(flanking_genomic_contexti) (barcodeᵢ)TGCGAGTCTTACAGCATTGC (SEQ ID NO:128)

Constant backbone sequence can be increased as the flanking genomic context is varied. For example, with 10 bp genomic flanking sequence on both sides:
GACGTTCTCACAGCAATTCGTACAGTCGACGTCGATTCGTGCT(barcodeᵢ)TTTG ACATTCTGCAATGT(flanking_genomic_contexti)(potential_off_target_siteᵢ)(flanking genomic_contextᵢ)(AAGTATGTATGCTTCGCGCAGTGCGACTTCGCAGCGCATCA CTTCA(barcodeᵢ)AGTAGCTGCGAGTCTTACAGCATTGC (SEQ ID NO:129)

### Other Library Generation Strategies:

incorporate population based SNPs into genomic sequences
generate libraries based on only coding DNA sequences
generate libraries of sites that are oncogene hotspots or tumor suppressor genes

The following are examples of methods using off-target libraries constructed using the above principles.

### Method for In Vitro Selection of Cleaved Library Members

### 1. Library Amplification

We amplify the oligonucleotide libraries using primers that bind to the constant flanking regions that are found in all library members. These primers contain 5′ prime overhangs that introduce additional length and a unique molecular identifier. The libraries are amplified using the following protocol using 2 μl of 5 nM input library.

| | |
|---|---|
| SV (21 of 5 nM input library) | 2 |
| Thermopol buffer | 5 |
| Taq Polym. | 0.25 |
| dNTP 10 mM | 1 |
| KP extension_new_fw* | 1 |
| KP extension_new_rev* | 1 |
| H2O | 39.75 |
| RV | 50 |
| PCR program | |
| cycles | 12 |
| ID 95 | 30 |
| D 95 | 20 |
| A 50 | 15 |
| E 68 | 1 |
| FE 68 | 30 min |

SV—Samples Volume
RV—Reaction Volume
*KP_extension_new_fw, Primer Sequence: GCTGACTAGACACTGCTATCACACTCTCTCANNNNNNNNAGACGTTCTCACAGCAATTCG (SEQ ID NO: 130)
*KP_extension_new_rev, Primer Sequence: GCGTAATCACTGATGCTTCGTAAATGAGACANNNNNNNNTGCAATGCTGTAAGACTCGCA (SEQ ID NO: 131)

### 2. DNA Purification:

DNA purification with AMPure magnetic beads at a sample:bead ratio of 0.9X according to manufacturer's protocol.

### 3. Enzymatic Incubation:

Incubation of 300 ng of the chip-synthesized library with protein of interest at varying enzyme concentrations and incubation times. In most cases (Cas9, Cas9HF, BE3, ABE) it is sufficient to perform an 1-2h incubation of the enzyme in activity buffer on 300 ng of oligonucleotide library at a molar ratio of 10:10:1 for protein, sgRNA and DNA substrate, respectively. Depending on the specific protein function, these parameters may need to be optimized.

### 4. Optional DNA Nicking:

Depending on the analyzed protein, enzymatic incubation may not result in the creation of a DNA double strand break (DSB). In the case of BE3 and ABE both enzymes merely nick on strand of DNA while base editing the other. By employing USER enzyme or Endonuclease V for BE3 and ABE, respectively, it is possible to convert this DNA nick into a staggered DSB (see FIGS. 8 and 9). To achieve this, bead-purified DNA from step 4 is incubated with USER enzyme or Endonuclease V for one hour at 37° C. in their respective activity buffer.

### 5. DNA Purification:

DNA purification with AMPure magnetic beads at a sample:bead ratio of 1.5X according to manufacturer's protocol.

### 6. Optional DNA Blunting:

If an additional nicking step 5 was required, the staggered DSB will be blunted by incubation with Phusion Polymerase for 20 min at 72° C. and then cooled to 4° C.

### 7. DNA Purification:

DNA purification with AMPure magnetic beads at a sample:bead ratio of 1.5X according to manufacturer's protocol.

### 8. Adapter Ligation:

Next, half functional Y-shape adapters are ligated to the blunted DNA from step 7. To achieve this, we supply adapter in 10-fold molar excess over library fragments and ligate using the NEB quick ligation kit, incubating the reaction at 25° C. for 10 min.

### 9. Gel Purification:

Next, we perform a gel purification of the ligation reaction by employing a 2.5% Agarose gel. The electrophoresis is performed at 120 Volt for 1 hour. After 1 hour the sample containing lanes are excised at around 180 bp fragment size and DNA is extracted using the Qiagen gel extraction kit according to manufacturer's protocol.

### 10. PCR-Amplification:

The eluate from step 9 is subsequently used as input for two PCR reactions that amplify the Protospacer-adjacent and PAM-adjacent site of cut library members). The primers used in this PCR contain 5′ overhangs that can be subsequently used to append Illumina sequencing barcodes. Optionally, QPCR can be performed to determine the minimum number of PCR cycles required. The PCRs are performed using the following parameters:

| | |
|---|---|
| Sample Volume | 6 |
| Phusion High Fidelity | 10 |
| Buffer 5X | |
| Phusion Polymerase | 0.5 |
| dNTP 10 mM | 1 |
| PrimerA | 2.5 |
| PrimerB | 2.5 |
| H2O | 27.5 |
| PCR program | |
| cycles | 25-35 |
| ID 98 | 30 |
| D 98 | 10 |
| A 65 | 20 |
| E 72 | 5 |
| FE 72 | 5 min |

### 11. DNA Purification:

DNA purification with AMPure magnetic beads at a sample:bead ratio of 1.5X according to manufacturer's protocol.

12. Quality Control using capillary electrophoresis:

Quality control is performed by examining the PCR products via capillary electrophoresis.

### 13. PCR-Based NGS Library Preparation:

Sequencing adapters are appended to the PCR products from step 12 by performing a PCR with primers containing Illumina sequencing adapters. The PCRs are performed using the following parameters:

| | |
|---|---|
| Sample Volume | 50 ng total |
| Phusion High Fidelity | 10 |
| Buffer 5X | |
| Phusion Polymerase | 0.5 |
| dNTP 10 mM | 1 |
| IndexPrimerA | 2.5 |
| IndexPrimerB | 2.5 |
| H2O | Ad 50 |
| PCR program | |
| cycles | 10 |
| ID 98 | 30 |
| D 98 | 10 |
| A 65 | 30 |
| E 72 | 35 |
| FE 72 | 10 min |

### 14. DNA Purification:

DNA purification with AMPure magnetic beads at a sample:bead ratio of 1.5X according to manufacturer's protocol.

### 15. Next Generation Sequencing on Illumina Sequencers:

The DNA libraries from step14 are quantified via digital droplet PCR and sequenced on Illumina sequencer's according to the manufacturer's protocol.

### Method for Enrichment of DNA Binding Sites by Pulldown

1) Resuspend Snap Capture Beads (NEB)
2) Pipette 80 uL of the beads to a new 1.5 mL Eppendorf tube
3) Place tube in a magnetic particle separator and discard the supernatant
4) Add 1 mL of Immobilization Buffer (20 mM HEPES, 150 mM NaCl, 0.5% Tween20, 1 mM DTT, pH 6.5) and vortex gently
5) Place tube in a magnetic particle separator and discard the supernatant
6) Prepare the protein: Add Engen Spy dCas9 (SNAP-tag) (NEB) (4.5 uL of 20 uM per pull down reaction) to 500 uL of Immobilization Buffer
7) Add the diluted protein to the beads and mix well via pipetting
8) Incubate for 1 hour shaking at room temperature
9) Place tube in a magnetic particle separator and discard the supernatant
10) Wash the beads. Add 1 mL of Immobilization Buffer, pipette mix well, and then place the tube in a magnetic particle separator and discard the supernatant
11) Repeat step 10 twice more for a total of 3 washes. Perform the last wash with Immobilization Buffer with 10 ug/mL Heparin
12) Resuspend the beads in 45 uL of immobilization buffer per pull down
13) Mix the following:

| | Amount for 1 Pull Down |
|---|---|
| Component | Reaction |
| Water | Add enough to make the |
| | final volume 60 uL after |
| | adding everything |
| | including 0.9 pmol of |
| | Library |
| 10X Immobilization | 6 uL |
| Buffer + | |
| 100 ug/mL Heparin | |
| gRNA | 3500 ng |
| Engen Spy dCas9 | 45 uL |
| (SNAP-tag) + | |
| Magnet Beads | |

14) Incubate for 25 deg C. for 10 min
15) Add 0.9 pmol of library
16) Incubate at 37 deg C. for 30 min
17) Place the tube on a magnetic bead separator and discard the supernatant
18) Wash the beads 5 times with 200 uL of Immobilization Buffer with 10 ug/mL Heparin
19) Add 50 uL of water and 2 uL of Proteinase K and incubate at room temperature for 10 min while shaking
20) Clean up the pulled down product with DNA purification beads (for example, Ampure) and elute in 10 uL of 0.1X Buffer EB (QIAgen)

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1-2. (canceled)

3. A method of identifying double stranded DNA sequences that are modified by a base editing enzyme, the method comprising:
(i) providing a plurality of linear dsDNA oligonucleotides of known sequences, each oligonucleotide having a 5′ end and a 3′ end and bearing two copies of a unique identifier sequence at or near both the 3′ and 5′ ends of the oligonucleotide, and a common sequence that is present in each one of the oligonucleotides in the plurality;
(ii) incubating the plurality linear dsDNA oligonucleotides in the presence of a base editing enzyme under conditions sufficient for modification to occur;
(iii) amplifying the oligonucleotides with a polymerase that converts edited base pairs to equal mixtures of canonical base pairs during DNA synthesis, optionally wherein a dATP nucleotide is incorporated across from dU or a dCTP nucleotide is incorporated across from dI, such that an oligonucleotide that has been modified by the base editing enzyme will be amplified as a mixture of the original barcode-linked sequence from the pre-treatment library and also a modified sequence that contains substitutions; and
(iv) determining the sequences of the amplified oligonucleotides, thereby identifying double stranded DNA sequences that are modified by the base editing enzyme.

4-7. (canceled)

8. The method of claim 3, wherein the linear dsDNA oligonucleotides comprise:
(i) a set of all potential off-target sequences in a reference genome bearing up to a certain number of mismatches relative to an identified on-target site (analogous to genomic DNA libraries);
(ii) a comprehensive set of potential off-target sites bearing up to a certain number of mismatches;
(iii) a library of potential off-target sequences present in a set of variant genomes from defined populations, optionally genomic DNA libraries designed to reflect DNA sequence variants present in a population of individuals; or
(iv) another relevant defined set of potential off-target sites, optionally oncogene hotspots or sequences from tumor suppressor genes.

9. The method of claim 3, wherein:
the linear DNA library members are first synthesized as individual single-stranded DNA sequences, optionally on high-density oligonucleotide arrays; and
the single-stranded DNA sequences are converted into double-stranded DNA molecules by priming against the common sequence, optionally before or after being released from the chip.

10. (canceled)

11. The method of claim 3, wherein the linear DNA library members comprise 1,000 to 10¹¹ different sequences.

12. The method of claim 3, wherein the pre enriched linear DNA library members comprise sequences that are 50 to 500 bp long.

13. The method of claim 3, wherein the base editing enzyme is a cytidine deaminase that converts deoxycytidine to deoxyuridine or an adenine base editing enzyme that converts deoxyadenine to deoxyinosine.

14. The method of claim 3, wherein the polymerase that converts edited base pairs to equal mixtures of canonical base pairs is a uracil tolerant polymerase that converts dU:dG base pairs to equal mixtures of dT:dA and dC:dG base pairs, or that converts dI:dT base pairs to equal mixtures of dA:dT and dG:dC base pairs.

15. (canceled)

16. The method of claim 3, wherein during the amplifying in step (iii), a dATP nucleotide is incorporated across from dU or a dCTP nucleotide is incorporated across from dI.

17. The method of claim 8, wherein the linear dsDNA oligonucleotides comprise a set of all potential off-target sequences in a reference genome bearing up to a certain number of mismatches relative to an identified on-target site (analogous to genomic DNA libraries).

18. The method of claim 8, wherein the linear dsDNA oligonucleotides comprise a comprehensive set of potential off-target sites bearing up to a certain number of mismatches.

19. The method of claim 8, wherein the linear dsDNA oligonucleotides comprise a library of potential off-target sequences present in a set of variant genomes from defined populations, optionally genomic DNA libraries designed to reflect DNA sequence variants present in a population of individuals.

20. The method of claim 8, wherein the linear dsDNA oligonucleotides comprise a defined set of potential off-target sites.

21. The method of claim 20, wherein the defined set of potential off target sites comprise oncogene hotspots or sequences from tumor suppressor genes.

22. The method of claim 9, wherein the linear DNA library members are first synthesized as individual single-stranded DNA sequences on high-density oligonucleotide arrays.

23. The method of claim 9, wherein the single stranded DNA sequences are converted into double-stranded DNA molecules by priming against the common sequence before being released from the chip.

24. The method of claim 9, wherein the single stranded DNA sequences are converted into double-stranded DNA molecules by priming against the common sequence after being released from the chip.


SEQUENCE LISTING (131 sequences)

SEQ ID NO: 1 - DNA, 23 bp, synthetic construct
  misc_feature 1..23: note = target sequence
  source 1..23: mol_type = other DNA
gcagatgtagtgtttccacaggg

SEQ ID NO: 2 - DNA, 23 bp, synthetic construct
  misc_feature 1..23: note = target sequence
  source 1..23: mol_type = other DNA
gaagatgtagtgtttccacaggg

SEQ ID NO: 3 - DNA, 23 bp, synthetic construct
  misc_feature 1..23: note = target sequence
  source 1..23: mol_type = other DNA
gccgatgtagtgtttccacaggg

SEQ ID NO: 4 - DNA, 23 bp, synthetic construct
  misc_feature 1..23: note = target sequence
  source 1..23: mol_type = other DNA
gcaaatgtagtgtttccacaggg

SEQ ID NO: 5 - DNA, 23 bp, synthetic construct
  misc_feature 1..23: note = target sequence
  source 1..23: mol_type = other DNA
gcagccgtagtgtttccacaggg

SEQ ID NO: 6 - DNA, 23 bp, synthetic construct
  misc_feature 1..23: note = target sequence
  source 1..23: mol_type = other DNA
gcagctatagtgtttccacaggg

SEQ ID NO: 7 - DNA, 23 bp, synthetic construct
  misc_feature 1..23: note = target sequence
  source 1..23: mol_type = other DNA
acagatttagggtttccacaggg

SEQ ID NO: 8 - DNA, 23 bp, synthetic construct
  misc_feature 1..23: note = target sequence
  source 1..23: mol_type = other DNA
gcagatgaagtgtaccccctggg

SEQ ID NO: 9 - DNA, 23 bp, synthetic construct
  misc_feature 1..23: note = target sequence
  source 1..23: mol_type = other DNA
gcagatgaggtcatcccacaggg

SEQ ID NO: 10 - DNA, 23 bp, synthetic construct
  misc_feature 1..23: note = target sequence
  source 1..23: mol_type = other DNA
acatatgaagtgtttggttaggc

SEQ ID NO: 11 - DNA, 23 bp, synthetic construct
  misc_feature 1..23: note = target sequence
  source 1..23: mol_type = other DNA
gcagatatagtgcgtccccaggg

SEQ ID NO: 12 - DNA, 23 bp, synthetic construct
  misc_feature 1..23: note = target sequence
  source 1..23: mol_type = other DNA
aagtgaggttgcctgccctgtct

SEQ ID NO: 13 - DNA, 23 bp, synthetic construct
  misc_feature 1..23: note = target sequence
  source 1..23: mol_type = other DNA
cctacctgaggctgaggaaggag

SEQ ID NO: 14 - DNA, 23 bp, synthetic construct
  misc_feature 1..23: note = target sequence
  source 1..23: mol_type = other DNA
ggtcacctacagcaccgagtgtg

SEQ ID NO: 15 - DNA, 23 bp, synthetic construct
  misc_feature 1..23: note = target sequence
  source 1..23: mol_type = other DNA
agctgaagaaggccaggtgtgag

SEQ ID NO: 16 - DNA, 23 bp, synthetic construct
  misc_feature 1..23: note = target sequence
  source 1..23: mol_type = other DNA
ctgtagcaggatgagccgcagac

SEQ ID NO: 17 - DNA, 12 bp, synthetic construct
  misc_feature 1..12: note = target sequence
  source 1..12: mol_type = other DNA
gctcaatgacgt

SEQ ID NO: 18 - DNA, 12 bp, synthetic construct
  misc_feature 1..12: note = target sequence
  source 1..12: mol_type = other DNA
gctcaatgacgt

SEQ ID NO: 19 - DNA, 17 bp, synthetic construct
  misc_feature 1..17: note = target sequence
  source 1..17: mol_type = other DNA
gagtccgagcagaagaa

SEQ ID NO: 20 - skipped

SEQ ID NO: 21 - DNA, 12 bp, synthetic construct
  misc_feature 1..12: note = target sequence
  source 1..12: mol_type = other DNA
gctcaatgacgt

SEQ ID NO: 22 - DNA, 23 bp, synthetic construct
  misc_feature 1..23: note = target sequence
  source 1..23: mol_type = other DNA
gagtccgagcagaagaagaaggg

SEQ ID NO: 23 - DNA, 23 bp, synthetic construct
  misc_feature 1..23: note = target sequence
  misc_feature 21: note = n is a, c, g, or t
  source 1..23: mol_type = other DNA
gagtccgagcagaagaagaangg

SEQ ID NO: 24 - DNA, 23 bp, synthetic construct
  misc_feature 1..23: note = target sequence
  source 1..23: mol_type = other DNA
ggaagcccttctgcagcaccaga

SEQ ID NO: 25 - DNA, 23 bp, synthetic construct
  misc_feature 1..23: note = target sequence
  source 1..23: mol_type = other DNA
ggaatcccttctgcagcatagtg

SEQ ID NO: 26 - DNA, 23 bp, synthetic construct
  misc_feature 1..23: note = target sequence
  source 1..23: mol_type = other DNA
ggaaacccctctgcagcaccagc

SEQ ID NO: 27 - DNA, 18 bp, synthetic construct
  misc_feature 1..18: note = target sequence
  source 1..18: mol_type = other DNA
ggaagcccttctaccaga

SEQ ID NO: 28 - DNA, 25 bp, synthetic construct
  misc_feature 1..25: note = target sequence
  source 1..25: mol_type = other DNA
ggaagcccttctgtagcacaccaga

SEQ ID NO: 29 - DNA, 24 bp, synthetic construct
  misc_feature 1..24: note = target sequence
  source 1..24: mol_type = other DNA
ggaagcccttctgtagccaccaga

SEQ ID NO: 30 - DNA, 27 bp, synthetic construct
  misc_feature 1..27: note = target sequence
  source 1..27: mol_type = other DNA
ggaatcccttctgcagccagcatagtg

SEQ ID NO: 31 - DNA, 25 bp, synthetic construct
  misc_feature 1..25: note = target sequence
  source 1..25: mol_type = other DNA
ggaatcccttctgcagcgcatagtg

SEQ ID NO: 32 - DNA, 20 bp, synthetic construct
  misc_feature 1..20: note = target sequence
  source 1..20: mol_type = other DNA
ggaatcccttctgcatagtg

SEQ ID NO: 33 - DNA, 20 bp, synthetic construct
  misc_feature 1..20: note = target sequence
  source 1..20: mol_type = other DNA
ggaaacccctctgcaccagc

SEQ ID NO: 34 - DNA, 24 bp, synthetic construct
  misc_feature 1..24: note = target sequence
  source 1..24: mol_type = other DNA
ggaaacccctctgcagcaaccagc

SEQ ID NO: 35 - DNA, 65 bp, synthetic construct
  misc_feature 1..65: note = target sequence
  source 1..65: mol_type = other DNA
ggaaacccctctgcagcagcaatggcaacaacgttgcgcaaactattaactggcgaactacttaa

SEQ ID NO: 36 - DNA, 24 bp, synthetic construct
  misc_feature 1..24: note = target sequence
  source 1..24: mol_type = other DNA
ggaaacccctctgcagccaccagc

SEQ ID NO: 37 - DNA, 22 bp, synthetic construct
  misc_feature 1..22: note = target sequence
  source 1..22: mol_type = other DNA
ggaaacccctctgcagcccagc

SEQ ID NO: 38 - DNA, 25 bp, synthetic construct
  misc_feature 1..25: note = target sequence
  source 1..25: mol_type = other DNA
ggaaacccctctgcagcgcaccagc

SEQ ID NO: 39 - DNA, 63 bp, synthetic construct
  misc_feature 1..63: note = target sequence
  source 1..63: mol_type = other DNA
ggaaacccctctgcagcggtgaaagaaagaatcgtcaactttggccatctcattgctaccagc

SEQ ID NO: 40 - DNA, 23 bp, synthetic construct
  misc_feature 1..23: note = target sequence
  source 1..23: mol_type = other DNA
gaggccgagcagaagaaagacgg

SEQ ID NO: 41 - DNA, 23 bp, synthetic construct
  misc_feature 1..23: note = target sequence
  source 1..23: mol_type = other DNA
aagtccgaggagaggaagaaagg

SEQ ID NO: 42 - DNA, 23 bp, synthetic construct
  misc_feature 1..23: note = target sequence
  source 1..23: mol_type = other DNA
gtcatcttagtcattacctgagg

SEQ ID NO: 43 - DNA, 23 bp, synthetic construct
  misc_feature 1..23: note = target sequence
  source 1..23: mol_type = other DNA
ggaatcccttctgcagcacctgg

SEQ ID NO: 44 - DNA, 22 bp, synthetic construct
  misc_feature 1..22: note = target sequence
  source 1..22: mol_type = other DNA
ttgccccacagggcagtaacgg

SEQ ID NO: 45 - DNA, 23 bp, synthetic construct
  misc_feature 1..23: note = target sequence
  source 1..23: mol_type = other DNA
gaacacaaagcatagactgcggg

SEQ ID NO: 46 - DNA, 23 bp, synthetic construct
  misc_feature 1..23: note = target sequence
  source 1..23: mol_type = other DNA
ggcccagactgagcacgtgatgg

SEQ ID NO: 47 - DNA, 23 bp, synthetic construct
  misc_feature 1..23: note = target sequence
  source 1..23: mol_type = other DNA
ggcactgcggctggaggtggggg

SEQ ID NO: 48 - DNA, 23 bp, synthetic construct
  misc_feature 1..23: note = target sequence
  source 1..23: mol_type = other DNA
ggctaaagaccatagactgtggg

SEQ ID NO: 49 - DNA, 23 bp, synthetic construct
  misc_feature 1..23: note = target sequence
  source 1..23: mol_type = other DNA
gggaataaatcatagaatcctgg

SEQ ID NO: 50 - DNA, 23 bp, synthetic construct
  misc_feature 1..23: note = target sequence
  source 1..23: mol_type = other DNA
acacacacacttagaatctgtgg

SEQ ID NO: 51 - DNA, 23 bp, synthetic construct
  misc_feature 1..23: note = target sequence
  source 1..23: mol_type = other DNA
ggtgagtgagtgtgtgcgtgtgg

SEQ ID NO: 52 - DNA, 23 bp, synthetic construct
  misc_feature 1..23: note = target sequence
  source 1..23: mol_type = other DNA
gtacaggagcaggagaagaatgg

SEQ ID NO: 53 - DNA, 23 bp, synthetic construct
  misc_feature 1..23: note = target sequence
  source 1..23: mol_type = other DNA
caaacggagcagaagaagaaagg

SEQ ID NO: 54 - DNA, 23 bp, synthetic construct
  misc_feature 1..23: note = target sequence
  source 1..23: mol_type = other DNA
gagcacgagcaagagaagaaggg

SEQ ID NO: 55 - DNA, 23 bp, synthetic construct
  misc_feature 1..23: note = target sequence
  source 1..23: mol_type = other DNA
gagtacaagcagatgaaaaacgg

SEQ ID NO: 56 - DNA, 23 bp, synthetic construct
  misc_feature 1..23: note = target sequence
  source 1..23: mol_type = other DNA
gagttagagcagaggaagagagg

SEQ ID NO: 57 - DNA, 23 bp, synthetic construct
  misc_feature 1..23: note = target sequence
  source 1..23: mol_type = other DNA
aagtccgggcaaaagaggaaagg

SEQ ID NO: 58 - DNA, 23 bp, synthetic construct
  misc_feature 1..23: note = target sequence
  source 1..23: mol_type = other DNA
gagtccaagcagtagaggaaggg

SEQ ID NO: 59 - DNA, 24 bp, synthetic construct
  misc_feature 1..24: note = target sequence
  source 1..24: mol_type = other DNA
cagtagtgagcagaagaagatagg

SEQ ID NO: 60 - DNA, 23 bp, synthetic construct
  misc_feature 1..23: note = target sequence
  source 1..23: mol_type = other DNA
gagttagagcagaagaagaaagg

SEQ ID NO: 61 - DNA, 23 bp, synthetic construct
  misc_feature 1..23: note = target sequence
  source 1..23: mol_type = other DNA
gtccaagagcaggagaagaaggg

SEQ ID NO: 62 - DNA, 23 bp, synthetic construct
  misc_feature 1..23: note = target sequence
  source 1..23: mol_type = other DNA
aagtccatgcagaagaggaaggg

SEQ ID NO: 63 - DNA, 23 bp, synthetic construct
  misc_feature 1..23: note = target sequence
  source 1..23: mol_type = other DNA
ggagtagagcagaggaagaaggg

SEQ ID NO: 64 - DNA, 23 bp, synthetic construct
  misc_feature 1..23: note = target sequence
  source 1..23: mol_type = other DNA
ggaactgagcaaaagaagatagg

SEQ ID NO: 65 - DNA, 23 bp, synthetic construct
  misc_feature 1..23: note = target sequence
  source 1..23: mol_type = other DNA
gaatccaagcagaagaagagaag

SEQ ID NO: 66 - DNA, 23 bp, synthetic construct
  misc_feature 1..23: note = target sequence
  source 1..23: mol_type = other DNA
gcgacagagcagaagaagaaggg

SEQ ID NO: 67 - DNA, 23 bp, synthetic construct
  misc_feature 1..23: note = target sequence
  source 1..23: mol_type = other DNA
gaagtagagcagaagaagaagcg

SEQ ID NO: 68 - DNA, 23 bp, synthetic construct
  misc_feature 1..23: note = target sequence
  source 1..23: mol_type = other DNA
gagtctaagcaggagaataaagg

SEQ ID NO: 69 - DNA, 23 bp, synthetic construct
  misc_feature 1..23: note = target sequence
  source 1..23: mol_type = other DNA
tgtccagagcagatgaagaatgg

SEQ ID NO: 70 - DNA, 23 bp, synthetic construct
  misc_feature 1..23: note = target sequence
  source 1..23: mol_type = other DNA
gaacatgagcagaagaagaggag

SEQ ID NO: 71 - DNA, 23 bp, synthetic construct
  misc_feature 1..23: note = target sequence
  source 1..23: mol_type = other DNA
aggccagagcaaaagaagagagg

SEQ ID NO: 72 - DNA, 23 bp, synthetic construct
  misc_feature 1..23: note = target sequence
  source 1..23: mol_type = other DNA
gtacaagagcaggagaagaaggg

SEQ ID NO: 73 - DNA, 23 bp, synthetic construct
  misc_feature 1..23: note = target sequence
  source 1..23: mol_type = other DNA
gagtcagggcagaagaagaaaat

SEQ ID NO: 74 - DNA, 23 bp, synthetic construct
  misc_feature 1..23: note = target sequence
  source 1..23: mol_type = other DNA
gagtaagagaagaagaagaaggg

SEQ ID NO: 75 - DNA, 23 bp, synthetic construct
  misc_feature 1..23: note = target sequence
  source 1..23: mol_type = other DNA
aagcccgagcagaagaagttgag

SEQ ID NO: 76 - DNA, 23 bp, synthetic construct
  misc_feature 1..23: note = target sequence
  source 1..23: mol_type = other DNA
gagtcctagcaggagaagaagag

SEQ ID NO: 77 - DNA, 22 bp, synthetic construct
  misc_feature 1..22: note = target sequence
  source 1..22: mol_type = other DNA
gaggggagcaaaagaaggaggg

SEQ ID NO: 78 - DNA, 23 bp, synthetic construct
  misc_feature 1..23: note = target sequence
  source 1..23: mol_type = other DNA
gagtcagagcaaaagaagtagtg

SEQ ID NO: 79 - DNA, 23 bp, synthetic construct
  misc_feature 1..23: note = target sequence
  source 1..23: mol_type = other DNA
gagtcagagcaaaagaagtagtg

SEQ ID NO: 80 - DNA, 23 bp, synthetic construct
  misc_feature 1..23: note = target sequence
  source 1..23: mol_type = other DNA
gagtctaagcagaagaagaagag

SEQ ID NO: 81 - DNA, 21 bp, synthetic construct
  misc_feature 1..21: note = target sequence
  source 1..21: mol_type = other DNA
cagggagaagaagaagaaggg

SEQ ID NO: 82 - DNA, 23 bp, synthetic construct
  misc_feature 1..23: note = target sequence
  source 1..23: mol_type = other DNA
gagttagagaagaagaagactgg

SEQ ID NO: 83 - DNA, 23 bp, synthetic construct
  misc_feature 1..23: note = target sequence
  source 1..23: mol_type = other DNA
acaatcgagcagcagaagaatgg

SEQ ID NO: 84 - DNA, 23 bp, synthetic construct
  misc_feature 1..23: note = target sequence
  source 1..23: mol_type = other DNA
gagtaggagcagatgaagagagg

SEQ ID NO: 85 - DNA, 23 bp, synthetic construct
  misc_feature 1..23: note = target sequence
  source 1..23: mol_type = other DNA
cagtatgagcaaaagaagaaaga

SEQ ID NO: 86 - DNA, 23 bp, synthetic construct
  misc_feature 1..23: note = target sequence
  source 1..23: mol_type = other DNA
gagtccatacagaggaagaaaag

SEQ ID NO: 87 - DNA, 21 bp, synthetic construct
  misc_feature 1..21: note = target sequence
  source 1..21: mol_type = other DNA
gaatgagcaaaagaagaaagc

SEQ ID NO: 88 - DNA, 23 bp, synthetic construct
  misc_feature 1..23: note = target sequence
  source 1..23: mol_type = other DNA
aatacagagcagaagaagaatgg

SEQ ID NO: 89 - DNA, 23 bp, synthetic construct
  misc_feature 1..23: note = target sequence
  source 1..23: mol_type = other DNA
aatacagagcagaagaagaatgg

SEQ ID NO: 90 - DNA, 21 bp, synthetic construct
  misc_feature 1..21: note = target sequence
  source 1..21: mol_type = other DNA
tagtgagcagaagaagagaga

SEQ ID NO: 91 - DNA, 23 bp, synthetic construct
  misc_feature 1..23: note = target sequence
  source 1..23: mol_type = other DNA
ttctccaagcagaagaagaagag

SEQ ID NO: 92 - DNA, 23 bp, synthetic construct
  misc_feature 1..23: note = target sequence
  source 1..23: mol_type = other DNA
gagtccatacaggagaagaaaga

SEQ ID NO: 93 - DNA, 23 bp, synthetic construct
  misc_feature 1..23: note = target sequence
  source 1..23: mol_type = other DNA
aggtgggagcagaagaagaaggg

SEQ ID NO: 94 - DNA, 23 bp, synthetic construct
  misc_feature 1..23: note = target sequence
  source 1..23: mol_type = other DNA
aaggcagagcagaggaagagagg

SEQ ID NO: 95 - DNA, 24 bp, synthetic construct
  misc_feature 1..24: note = target sequence
  source 1..24: mol_type = other DNA
gaggcacaagcaaaagaagaaaag

SEQ ID NO: 96 - DNA, 23 bp, synthetic construct
  misc_feature 1..23: note = target sequence
  source 1..23: mol_type = other DNA
gaagtagagcagaagaagaagcg

SEQ ID NO: 97 - DNA, 16 bp, synthetic construct
  misc_feature 1..16: note = target sequence
  source 1..16: mol_type = other DNA
ctatcttaaggtagtc

SEQ ID NO: 98 - DNA, 16 bp, synthetic construct
  misc_feature 1..16: note = target sequence
  source 1..16: mol_type = other DNA
actctcttaaggtagc

SEQ ID NO: 99 - DNA, 16 bp, synthetic construct
  misc_feature 1..16: note = target sequence
  source 1..16: mol_type = other DNA
ctatcttaaggtagcc

SEQ ID NO: 100 - DNA, 15 bp, synthetic construct
  misc_feature 1..15: note = target sequence
  source 1..15: mol_type = other DNA
ctaccttaaggtagt

SEQ ID NO: 101 - DNA, 15 bp, synthetic construct
  misc_feature 1..15: note = target sequence
  source 1..15: mol_type = other DNA
ctaccttaagggagc

SEQ ID NO: 102 - DNA, 15 bp, synthetic construct
  misc_feature 1..15: note = target sequence
  source 1..15: mol_type = other DNA
ctatcttaagggagc

SEQ ID NO: 103 - DNA, 15 bp, synthetic construct
  misc_feature 1..15: note = target sequence
  source 1..15: mol_type = other DNA
ctcccttaagggagc

SEQ ID NO: 104 - DNA, 16 bp, synthetic construct
  misc_feature 1..16: note = target sequence
  source 1..16: mol_type = other DNA
ctatcttaaggtaggc

SEQ ID NO: 105 - DNA, 16 bp, synthetic construct
  misc_feature 1..16: note = target sequence
  source 1..16: mol_type = other DNA
ctctcttaagggagcc

SEQ ID NO: 106 - DNA, 16 bp, synthetic construct
  misc_feature 1..16: note = target sequence
  source 1..16: mol_type = other DNA
ctctcttaaggtagct

SEQ ID NO: 107 - DNA, 15 bp, synthetic construct
  misc_feature 1..15: note = target sequence
  source 1..15: mol_type = other DNA
ctcccttaaggtagt

SEQ ID NO: 108 - DNA, 16 bp, synthetic construct
  misc_feature 1..16: note = target sequence
  source 1..16: mol_type = other DNA
ctctcttaaggtagtc

SEQ ID NO: 109 - DNA, 16 bp, synthetic construct
  misc_feature 1..16: note = target sequence
  source 1..16: mol_type = other DNA
ctctcttaagatagcc

SEQ ID NO: 110 - DNA, 15 bp, synthetic construct
  misc_feature 1..15: note = target sequence
  source 1..15: mol_type = other DNA
ctaccttaaggtagc

SEQ ID NO: 111 - DNA, 16 bp, synthetic construct
  misc_feature 1..16: note = target sequence
  source 1..16: mol_type = other DNA
ctcccttaaggtagtc

SEQ ID NO: 112 - DNA, 16 bp, synthetic construct
  misc_feature 1..16: note = target sequence
  source 1..16: mol_type = other DNA
ctctcataaggtagtc

SEQ ID NO: 113 - DNA, 16 bp, synthetic construct
  misc_feature 1..16: note = target sequence
  source 1..16: mol_type = other DNA
ctctcataaggtagcc

SEQ ID NO: 114 - DNA, 16 bp, synthetic construct
  misc_feature 1..16: note = target sequence
  source 1..16: mol_type = other DNA
ctctgttaaggtagtc

SEQ ID NO: 115 - DNA, 15 bp, synthetic construct
  misc_feature 1..15: note = target sequence
  source 1..15: mol_type = other DNA
ctcccttaagagagc

SEQ ID NO: 116 - DNA, 16 bp, synthetic construct
  misc_feature 1..16: note = target sequence
  source 1..16: mol_type = other DNA
ctcccttaaggtagcc

SEQ ID NO: 117 - DNA, 16 bp, synthetic construct
  misc_feature 1..16: note = target sequence
  source 1..16: mol_type = other DNA
ctcccttaaggtagac

SEQ ID NO: 118 - DNA, 15 bp, synthetic construct
  misc_feature 1..15: note = target sequence
  source 1..15: mol_type = other DNA
ctctcttaaggtagt

SEQ ID NO: 119 - DNA, 15 bp, synthetic construct
  misc_feature 1..15: note = target sequence
  source 1..15: mol_type = other DNA
ctatcttaaggtagt

SEQ ID NO: 120 - DNA, 15 bp, synthetic construct
  misc_feature 1..15: note = target sequence
  source 1..15: mol_type = other DNA
ctctcttaagggagc

SEQ ID NO: 121 - DNA, 15 bp, synthetic construct
  misc_feature 1..15: note = target sequence
  source 1..15: mol_type = other DNA
ctatcttaagggagt

SEQ ID NO: 122 - DNA, 16 bp, synthetic construct
  misc_feature 1..16: note = target sequence
  source 1..16: mol_type = other DNA
ctctgttaaggtagcc

SEQ ID NO: 123 - DNA, 16 bp, synthetic construct
  misc_feature 1..16: note = target sequence
  source 1..16: mol_type = other DNA
ctctcttaaggtaggc

SEQ ID NO: 124 - DNA, 15 bp, synthetic construct
  misc_feature 1..15: note = target sequence
  source 1..15: mol_type = other DNA
ctctcttaaggtagc

SEQ ID NO: 125 - DNA, 15 bp, synthetic construct
  misc_feature 1..15: note = target sequence
  source 1..15: mol_type = other DNA
ctatcttaaggtagc

SEQ ID NO: 126 - DNA, 15 bp, synthetic construct
  misc_feature 1..15: note = target sequence
  source 1..15: mol_type = other DNA
ctctcttaagagagc

SEQ ID NO: 127 - DNA, 153 bp, synthetic construct
  misc_feature 1..153: note = library backbone sequence
  misc_feature 43..44: note = positions 43 and 44 are non-consecutive residues separated by a barcode
  misc_feature 71..72: note = positions 71 and 72 are non-consecutive residues separated by a potential off target site
  misc_feature 127..128: note = positions 127 and 128 are non-consecutive residues separated by a barcode
  source 1..153: mol_type = other DNA
gacgttctcacagcaattcgtacagtcgacgtcgattcgtgcttttgacattctgcaattgcacacagcgttgcagactgtaagtatgtatgcttcgcgcagtgcgacttcgcagcgcatcacttcaagtagctgcgagtcttacagcattgc

SEQ ID NO: 128 - DNA, 41 bp, synthetic construct
  misc_feature 1..41: note = library backbone sequence
  misc_feature 21..22: note = positions 21 and 22 are non-consecutive residues separated by a barcode, flanking genomic context, potential_off_target_site, flanking_genomic_context, barcode
  source 1..41: mol_type = other DNA
gacgttctcacagcaattcgttgcgagtcttacagcattgc

SEQ ID NO: 129 - DNA, 133 bp, synthetic construct
  misc_feature 1..133: note = library backbone sequence
  misc_feature 43..44: note = positions 43 and 44 are non-consecutive residues separated by a barcode
  misc_feature 61..62: note = positions 61 and 62 are non-consecutive residues separated by a flanking_genomic_context, potential_off_target_sitei, and a flanking_genomic_context
  misc_feature 106..107: note = positiions 106 and 107 are non-consecutive residues separated by a barcode
  source 1..133: mol_type = other DNA
gacgttctcacagcaattcgtacagtcgacgtcgattcgtgcttttgacattctgcaatgtaagtatgtatgcttcgcgcagtgcgacttcgcagcgcatcacttcaagtagctgcgagtcttacagcattgc

SEQ ID NO: 130 - DNA, 60 bp, synthetic construct
  misc_feature 1..60: note = synthetic primer
  misc_feature 32..39: note = n is a, c, g, or t
  source 1..60: mol_type = other DNA
gctgactagacactgctatcacactctctcannnnnnnnagacgttctcacagcaattcg

SEQ ID NO: 131 - DNA, 60 bp, synthetic construct
  source 1..60: mol_type = other DNA
gcgtaatcactgatgcttcgtaaatgagacannnnnnnntgcaatgctgtaagactcgca